(19) United States
(12) Patent Application Publication
(10) Pub. No.: US 2007/0027535 A1
(43) Pub. Date: Feb. 1, 2007

(54) Implantable thromboresistant valve

(75) Inventors: James D. Purdy JR., Lafayette, IN (US); Charles W. Agnew, West Lafayette, IN (US); Brian C. Case, Lake Villa, IL (US); Ram H. Paul JR., Bloomington, IN (US)

Correspondence Address: BRINKS HOFER GILSON & LIONE/INDY/COOK, ONE INDIANA SQUARE, SUITE 1600, INDIANAPOLIS, IN, 46204-2033, US

(73) Assignee: Cook Incorporated, Bloomington, IN (US)

(21) Appl. No.: 11/494,424

(22) Filed: Jul. 27, 2006

Related U.S. Application Data
(60) Provisional application No. 60/703,217, filed on Jul. 28, 2005.
(60) Provisional application No. 60/780,443, filed on Mar. 8, 2006.

(51) Int. Cl. A61F 2/24; A61F 2/06

(52) U.S. Cl. 623/2.18; 623/2.42; 623/1.24

(57) ABSTRACT

Medical devices for implantation within a body vessel comprising a thromboresistant material are provided. The thromboresistant material preferably comprises a biocompatible polyurethane, a remodelable material, a bioactive agent or any combination thereof. The medical device can be a prosthetic valve comprising a thromboresistant material. The medical device can also comprise a support frame with one or more valve leaflets attached to the support frame.

### RELATED APPLICATIONS

This application claims the benefit of both U.S. Provisional Patent Application Ser. No. 60/703,217, filed Jul. 28, 2005 and entitled “IMPLANTABLE THROMBORESISTANT VALVE,” as well as U.S. Provisional Patent Application Ser. No. 60/780,443, filed Mar. 8, 2006 and entitled “IMPLANTABLE THROMBORESISTANT VALVE,” both of which are incorporated herein by reference in their entirety.

### TECHNICAL FIELD

The present invention relates to medical devices for implantation in a body vessel. More particularly, the present invention relates to implantable medical device with thromboresistant properties.

### BACKGROUND

Various implantable medical devices are advantageously inserted within various body vessels, for example to improve or replace the function of valves therein. For example, native valves within the heart and veins function to regulate blood flow within the body. Heart valves positioned within the heart direct the flow of blood to and from other organs and pump oxygenated blood to the rest of the body. Venous valves are typically bicuspid valves positioned at varying intervals within veins to permit substantially unidirectional blood to flow toward the heart.

Endovascular prosthesis can be implanted to treat various conditions. Stent grafts can be implanted to strengthen a blood vessel wall in the location of an aneurysm, or to open an occlusion in a blood vessel. Prosthetic valves can be implanted in various body passages to replace natural valves that are defective or diseased. Valves can also be implanted in and near the heart and at various positions within the venous system, including the implantation of prosthetic venous valves in the femoral and popliteal veins. Prosthetic cardiac valves have been used to replace the native cardiac valves within the heart using percutaneous approaches. Another type of prosthetic valve is a prosthetic venous valve. Prosthetic valves have also been implanted in veins to promote the flow of blood back to the heart. Blood pressure, as provided by heart activity via the arteries, is normally sufficient to maintain the flow of blood in one direction. The blood pressure in the veins can be much lower than in the arteries principally due to their distance from the heart. Venous valves function to limit the backflow of blood through the veins. Numerous such venous valves are located throughout the venous system and are particularly important to maintaining proper blood flow in the lower extremities. Venous valves can become incompetent and lead to chronic venous insufficiency. Various techniques have been developed for treating incompetent venous valves including valvuloplasty, transplantation, and replacement with a prosthetic valve. These techniques include both open and percutaneous approaches.

Minimally invasive techniques and instruments for placement of intraluminal medical devices have been developed to treat and repair undesirable conditions within body vessels, including treatment of conditions that affect blood flow such as venous valve insufficiency. Various percutaneous methods of implanting medical devices within the body using intraluminal transcatheter delivery systems can be used to treat a variety of conditions. One or more intraluminal medical devices can be introduced to a point of treatment within a body vessel using a delivery catheter device passed through the vasculature communicating between a remote introductory location and the implantation site, and released from the delivery catheter device at the point of treatment within the body vessel. Intraluminal medical devices can be deployed in a body vessel at a point of treatment and the delivery device subsequently withdrawn from the vessel, while the medical device retained within the vessel to provide sustained improvement in vascular valve function or to increase vessel patency.

Inhibiting or preventing thrombosis and platelet deposition on an implantable device within the body is important in promoting continued function of the medical device within the body, particularly within blood vessels. Post-implantation thrombosis and platelet deposition on surfaces of implantable medical devices prosthesis undesirably reduce the patency rate of many implantable medical devices. For example, thrombosis and platelet deposition within an endovascular prosthesis may occlude the conduit defined by the endovascular prosthesis or compromise the function of an implanted valve by limiting the motion or responsiveness of moveable portions of the device such as valve leaflets. Many factors contribute to thrombosis and platelet deposition on the surfaces of implanted prosthesis. The properties of the material or materials forming the endovascular prosthesis are believed to be one important factor that can contribute to the likelihood of undesirable levels of post-implantation thrombus formation or platelet deposition on the implanted device. Incorporation of bioactive materials that inhibit platelet deposition and promote tissue ingrowth, such as growth factors, can promote formation of a non-thrombogenic tissue coating over portions of a prosthetic implant. The formation of blood clots, or thrombus, on the surface of an endovascular prosthesis can both degrade the intended performance of the prosthesis and even undesirably restrict or occlude desirable fluid flow within a body vessel.

What is needed are implantable medical devices having thromboresistant properties. The implantable medical devices provided herein comprise a thromboresistant material, a thromboresistant agent, or a combination thereof. Preferably, the medical devices are suitable for use as percutaneously implantable valves, such as venous valves or heart valves, that can be delivered using a minimally invasive catheter-based delivery system.

### SUMMARY

The present invention relates to an implantable medical device for placement within a body passage. The medical device is preferably an implantable valve comprising a biocompatible thromboresistant material to mitigate thrombus formation. The thromboresistant material is preferably a biocompatible polyurethane material. The implantable medical device may optionally include one or materials that promote the deposition of native endothelial cells on at least a portion of the medical device. The biocompatible polyurethane material desirably comprises a growth factor to promote deposition of endothelial cells on a surface of the medical device, for example by remodeling processes.

In a first embodiment, a frameless implantable valve is provided. A portion of the frameless implantable valve is moveable in response to fluid flow within a body vessel, so as to permit fluid flow in a first direction while substantially preventing fluid flow in the opposite direction. The moveable portion of the frameless valve preferably comprises a thromboresistant material, a thromboresistant bioactive agent, or a combination thereof. A frameless implantable valve have various configurations. For example, a frameless valve can be formed by securing a valve leaflet within a body vessel. The valve leaflet can comprise a moveable portion of a sheet of thromboresistant material that releasably contacts a portion of a body vessel wall to regulate fluid flow therein. The sheet preferably has thickened edges and anchored to the wall of a body vessel.

In a second embodiment, an implantable medical device comprises a thromboresistant material attached to a support means for providing structural support to the thromboresistant material. The support means can be formed from any suitable structure that maintains an attached thromboresistant material in a desired position, orientation or range of motion to perform a desired function. Preferably, the support means permits the thromboresistant material to perform a valving function to regulate fluid flow within a body vessel. More preferably, the support means is a support frame attached to one or more thromboresistant valve leaflets. The support means is preferably a substantially cylindrical implantable frame defining a central longitudinal lumen. The implantable frame preferably defines a substantially cylindrical or elliptical lumen providing a conduit for fluid flow. In another aspect, the implantable medical device comprises a means for regulating fluid flow coupled to an implantable frame. The means for regulating fluid flow is preferably a moveable valve surface formed at least in part from a thromboresistant material. In some embodiments, the fluid can flow through interstitial spaces between strut or bend portions of the frame, while other embodiments provide for fluid flow through a lumen defined along a substantially cylindrical interior surface of the frame. For example, the support means can be an implantable substantially cylindrical frame comprising a plurality of interconnecting struts and bends defining openings in the cylindrical outer surface of the frame having any suitable shape and pattern. Alternatively, the support means can be a continuous tube, with or without openings in the outer surface area of the frame, formed from a biocompatible material, such as a polymer, or a tube of woven fabric.

In a third embodiment, an implantable valve comprising an adhesion promoting body vessel contact region is provided. The adhesion promoting region of the implantable valve is adapted to promote adhesion of the contact region of the implantable valve to the surface of a body vessel, preferably by promoting the ingrowth of cells and tissue from the body vessel into the contact region of the implanted valve. The adhesion promoting region of the implantable valve can comprise a remodelable material, a porous thromboresistant polyurethane polymer, a tissue growth promoting bioactive agent such as a growth factor, a thromboresistant bioactive agent, or any combination thereof. Preferred materials for forming an adhesion promoting region include: porous forms of a biocompatible polyurethane, an extracellular matrix material, and combinations thereof. Any implantable device, including a frameless valve and implantable valves comprising a support frame, can comprise one or more adherence promoting region.

In a fourth embodiment, the medical device comprises a surface formed from a biocompatible polyurethane material comprising a growth factor and optionally further comprising a remodelable material. In a first aspect, the fourth embodiment provides valve leaflets comprising a first layer formed from a biocompatible polyurethane attached to a remodelable material. The remodelable material can be confined to the edges where the valve leaflet is attached to the support frame, for example to form an adhesion promoting body vessel contact region. Remodelable material can also be mixed with the biocompatible polyurethane. The remodelable material preferably includes one or more growth factors. The remodelable material can also form a second layer laminated to the first layer of biocompatible polyurethane. In a second aspect, the fourth embodiment provides valve leaflets comprising a first layer formed from a sheet of remodelable material in contact with a biocompatible polyurethane. The biocompatible polyurethane can be laminated to, mixed with or deposited on a portion of the remodelable material. Preferably, the biocompatible polyurethane contacting the remodelable material has a porous structure to provide for tissue ingrowth and tissue access to growth factors within the remodelable material. The remodelable material is preferably small intestine submucosa (SIS).

In a fifth embodiment, methods for making a prosthetic valve for placement within a body passage are also provided. Preferably, the prosthetic valve comprises a thromboresistant material. According to one preferred method, a solution comprising a dissolved thromboresistant material is sprayed and dried on a mandrel. The solution of thromboresistant material preferably comprises a suitable solvent, a biocompatible polyurethane and a surface modifying agent. The mandrel is preferably configured to provide a desirable leaflet shape. One or more leaflets can be formed by coating and drying one or more layers of the solution of the thromboresistant material on the surface of the mandrel. The thromboresistant material can be attached to a support frame by spray coating the solution of the thromboresistant material onto the support frame. An assembly comprising an implantable support frame and a mandrel is spray coated with the solution of the thromboresistant material to form a prosthetic valve comprising one or more leaflets formed from the thromboresistant material. The spray coated assembly can be subsequently dried to form leaflets attached to the implantable frame. Alternatively, an assembly comprising an implantable support frame and a mandrel is dip coated with the solution of the thromboresistant material to form a prosthetic valve comprising one or more leaflets formed from the thromboresistant material. Preferably, an implantable valve can be formed by dipping a rotating assembly and dried upon removal from the solution to form leaflets attached to the implantable frame. Multiple layers of the solution of the thromboresistant material can be coated over the mandrel, the implantable frame, or both. Multiple layers of the solution of the thromboresistant material can be coated over the mandrel, the implantable frame, or both.

The medical device preferably comprises a radially expandable frame and a thromboresistant material attached to the frame. The medical device is preferably an implantable valve comprising one or more valve leaflets attached to the implantable frame. The one or more valve leaflets can be configured and positioned to regulate fluid flow through the implanted medical device. The implantable valve preferably comprises a valve orifice moveable to regulate fluid flow through the valve. The valve orifice can be formed by moveable portions of an implantable frame, by flexible free edges of a flexible material attached to the implantable frame, by a portion of the body vessel, or any combination thereof. Preferred implantable valve structures comprise two or three valve leaflets, although valves can comprise more or fewer leaflets. Preferably, a valve leaflet comprises a thromboresistant material or thromboresistant bioactive agent and is moveable in response to fluid flow within the frame lumen to regulate fluid flow in a substantially unidirectional manner therethrough. Optionally, the implantable frame can also comprise a thromboresistant material or thromboresistant bioactive agent. The valve leaflets can have a uniform thickness or a thickness that varies at different positions along the valve leaflet. For example, a valve leaflet can be thicker near points of attachment to a support frame, and thinner near a valve orifice region.

The invention includes other embodiments within the scope of the claims, and variations of all embodiments. Additional understanding of the invention can be obtained by referencing the detailed description of embodiments of the invention, below, and the appended drawings.

### BRIEF DESCRIPTION OF THE DRAWINGS

FIG. 1A is a perspective view of a frameless prosthetic valve that is an invertible frameless membrane prosthetic valve in a first configuration; FIG. 1B is a perspective view of the frameless prosthetic valve shown in FIG. 1A in a second configuration.

FIG. 2A is a top view of a prosthetic valve comprising two valve leaflets attached to a self-expanding support frame; FIG. 2B is a side view of the prosthetic valve shown in FIG. 2A; FIG. 2C is a perspective view of the prosthetic valve shown in FIG. 2A and FIG. 2B; FIG. 2D is a cross sectional view along the segment A-A′ shown in FIG. 2A; FIG. 2E is a cross sectional view along the segment B-B′ shown in FIG. 2B; FIG. 2F is an end view of the prosthetic valve shown in FIG. 2A, FIG. 2B and FIG. 2C.

FIG. 3A is a second implantable valve comprising a pair of valve leaflets and a support frame; FIG. 3B is the implantable valve of FIG. 3A, further comprising an adhesion promoting body vessel contact region.

FIG. 4 shows an implantable valve comprising an outer sleeve enclosing the implantable valve of FIG. 1A.

FIG. 5A schematically indicates a mandrel shaped for forming a pair of valve leaflets around the distal portion; FIG. 5B shows a side view of the mandrel of FIG. 5A; FIG. 5C illustrates spray coating of the distal mandrel portion with a solution of a biocompatible polyurethane; FIG. 5D shows the placement of a radially expandable support frame over the coated mandrel shown in FIG. 5A; FIG. 5E shows a radially expandable support frame over the distal end of the mandrel and FIG. 5F shows a cross section of the polyurethane coating joined to a cross section of the radially expandable support frame.

FIG. 6A shows the dipping of a mandrel into a solution comprising a coatable polyurethane; FIG. 6B shows the coating of the mandrel in the solution; FIG. 6C shows the placement of a radially expandable frame over a coated mandrel; and FIG. 7 shows removal of a valve from a mandrel.

### DETAILED DESCRIPTION

The following detailed description and appended drawings describe and illustrate various exemplary embodiments of the invention. The description and drawings serve to enable one skilled in the art to make and use the invention.

As used herein the terms “comprise(s),” “include(s),” “having,” “has,” “contain(s),” and variants thereof, are intended to be open-ended transitional phrases, terms, or words that do not preclude the possibility of additional acts or structure.

The term “about” used with reference to a quantity includes variations in the recited quantity that are equivalent to the quantity recited, for instance an amount that is insubstantially different from a recited quantity for an intended purpose or function.

As used herein, the term “implantable” refers to an ability of a medical device to be positioned at a location within a body, such as within a body vessel. Furthermore, the terms “implantation” and “implanted” refer to the positioning of a medical device at a location within a body, such as within a body vessel.

As used herein, the term “body vessel” means any tube-shaped body passage lumen that conducts fluid, including but not limited to blood vessels such as those of the human vasculature system, billiary ducts and ureteral passages.

As used herein, “endolumenally,” “intraluminally” or “transluminal” all refer synonymously to implantation placement by procedures wherein the prosthesis is advanced within and through the lumen of a body vessel from a remote location to a target site within the body vessel. In vascular procedures, a medical device will typically be introduced “endovascularly” using a catheter over a guidewire under fluoroscopic guidance. The catheters and guidewires may be introduced through conventional access sites to the vascular system, such as through the femoral artery, or brachial and subclavian arteries, for access to the coronary arteries.

An “upstream” direction within a vein is away from the heart; a “downstream” direction within a vein is toward the heart.

As used herein, the term “body vessel” means any body passage lumen that conducts fluid, including but not limited to blood vessels, esophageal, intestinal, billiary, urethral and ureteral passages.

The term “alloy” refers to a substance composed of two or more metals or of a metal and a nonmetal intimately united, for example by chemical or physical interaction. Alloys can be formed by various methods, including being fused together and dissolving in each other when molten, although molten processing is not a requirement for a material to be within the scope of the term “alloy.” As understood in the art, an alloy will typically have physical or chemical properties that are different from its components.

The term “mixture” refers to a combination of two or more substances in which each substance retains its own chemical identity and properties.

The terms “frame” and “support frame” are used interchangeably herein to refer to a structure that can be implanted, or adapted for implantation, within the lumen of a body vessel.

The present invention relates to implantable medical devices for placement within a body passage. The implantable medical device preferably comprises one or more thromboresistant materials. Preferably, the thromboresistant material is a biocompatible polyurethane material comprising a surface modifying agent, as described herein. The medical device is preferably a percutaneously implantable valve comprising a valve leaflet formed from thromboresistant material that can include one or more materials selected from the group consisting of: remodelable materials, growth factors, and thromboresistant bioactive agents. The implantable medical device can have any suitable configuration to perform a desired function, but preferably can function as an implantable valve adapted for implantation in a vein or within a heart.

Implantable Prosthetic Valves

Certain non-limiting examples of valve embodiments are provided herein to illustrate selected features of the medical devices relating to component frames. Medical devices can comprise the frame embodiments discussed below, and combinations, variations or portions thereof, as well as other frame configurations. Medical devices comprising various frames in combination with material suitable to form a leaflet attached thereto are also within the scope of some embodiments of the invention.

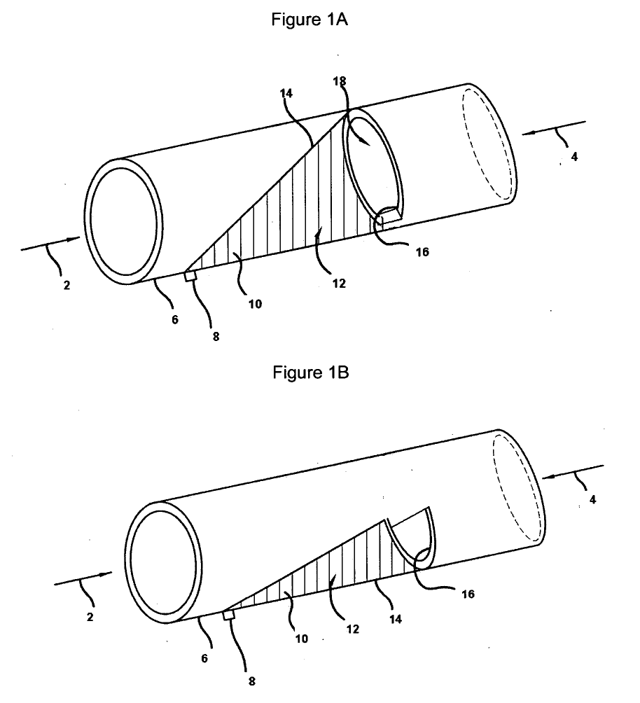

In a first embodiment, a frameless implantable valve is provided. A portion of the frameless implantable valve is moveable in response to fluid flow within a body vessel, so as to permit fluid flow in a first direction while substantially preventing fluid flow in the opposite direction. The moveable portion of the frameless valve preferably comprises a thromboresistant material, a thromboresistant bioactive agent, or a combination thereof. A frameless implantable valve have various configurations. FIG. 1A and FIG. 1B show an invertible frameless membrane prosthetic valve 12 within a segment of a body vessel 6. The invertible frameless membrane prosthetic valve 12 is formed from a sheet of thromboresistant material 10, and can be attached to the wall of a body vessel 6 by any suitable means, including an anchoring element 8 embedded within the wall of the body vessel. The anchoring element 8 can be any suitable structure configured to embed within the wall of a body vessel lumen, such as a barb or a suture. The thromboresistant material 10 has a first surface 14 and a second surface 16, and is sufficiently flexible to bend in response to fluid moving through the body vessel 6. In FIG. 1A, the prosthetic valve 12 forms a first configuration wherein fluid flow in a retrograde direction 4 is substantially blocked when the fluid contacts the second surface 16, resulting in the formation of a sinus pocket 18. In FIG. 1B, the invertible frameless membrane prosthetic valve 12 forms an inverted configuration with respect to the first configuration, wherein fluid flow in the antegrade direction 2 is permitted by contacting the second surface 16 and collapsing the valve 12 against the wall of the body vessel 6. Accordingly, fluid is permitted to move in the antegrade direction 2, but not in the retrograde direction 4. Preferably, the edges of the invertible frameless membrane prosthetic valve 12 are thicker than the central portion of the valve surface 14 so as to provide a stiffening of the edges relative to the center of the leaflet that promotes movement of the leaflet between the inverted and everted configurations. Preferably, the invertible frameless membrane prosthetic valve defines a portion of a cone. Preferably, the invertible frameless membrane prosthetic valves 12 include an anchoring element 8 adjacent a vertex of the cone.

Optionally, two or more invertible frameless membrane prosthetic valves 12 can be implanted within a body vessel. In one embodiment, two or more invertible frameless membrane prosthetic valves 12 can be implanted symmetrically within a body vessel. For example, two or more invertible frameless membrane prosthetic valves 12 can be implanted across from each other so that the first side 14 of each valve opposably define a valve orifice. Preferably, a plurality of invertible frameless membrane prosthetic valves 12 can be positioned within a body vessel. The plurality of invertible frameless membrane prosthetic valves 12 are more preferably symmetrically implantable in a body lumen and invertibly deformable between an inverted position and an everted position, wherein the invertible frameless membrane prosthetic valves 12 are moveable between an inverted configuration and an everted configuration in response to the direction of fluid flow through the lumen. Preferably, the invertible frameless membrane prosthetic valves 12 are invertible relative to a radial direction of a body vessel lumen, and are deformable by fluid flow in the body vessel lumen.

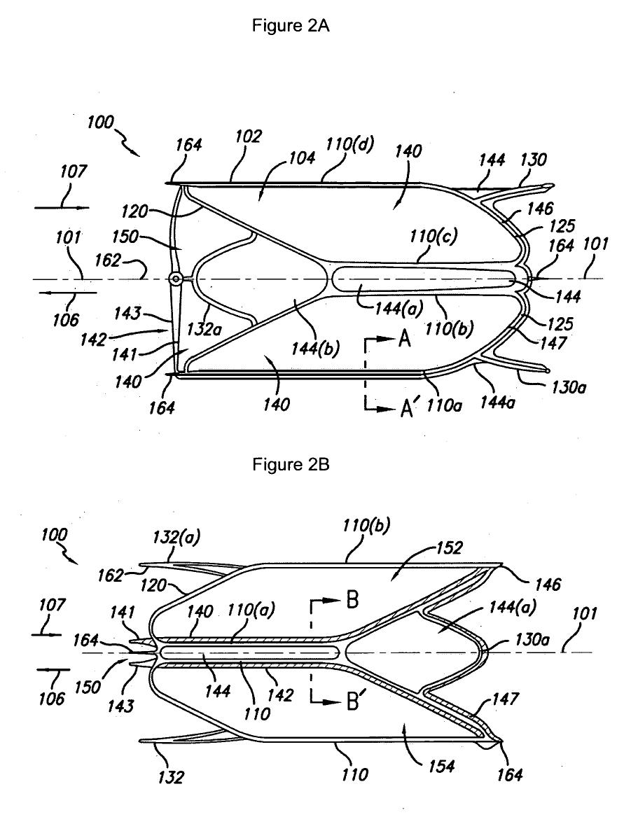

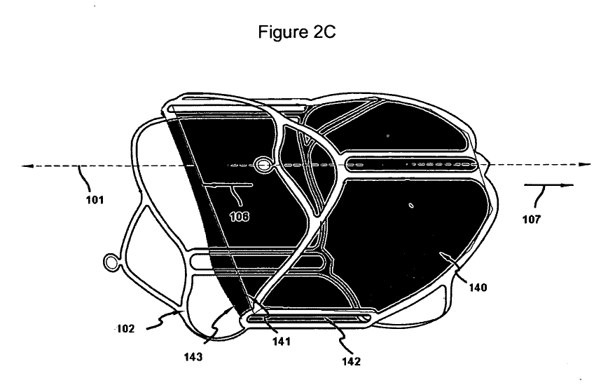

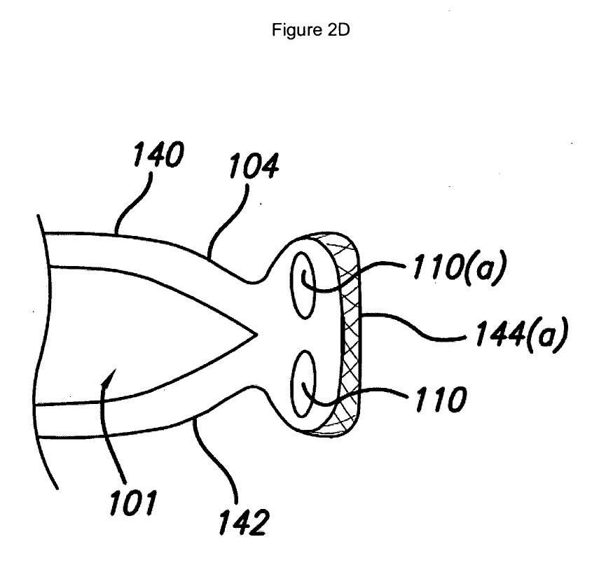

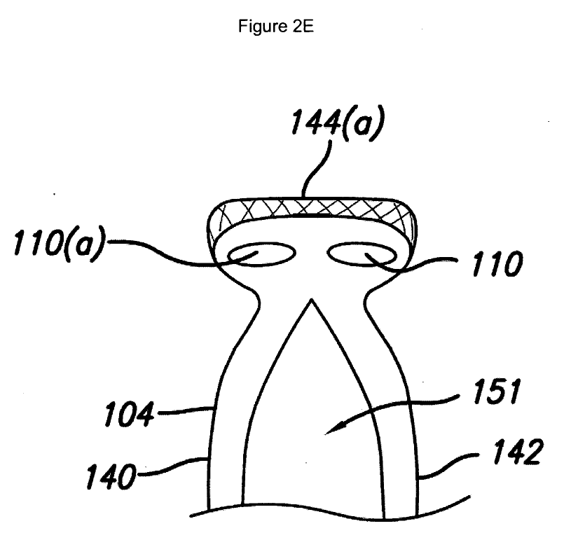

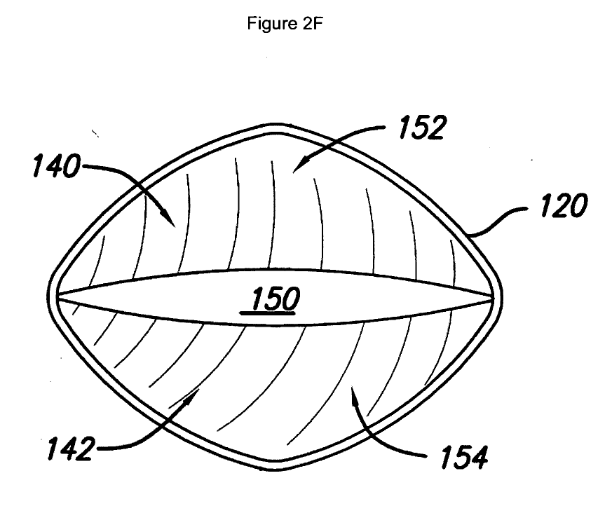

In a second embodiment, an implantable medical device comprises a thromboresistant material attached to a support means for providing structural support to the thromboresistant material. The support means can be formed from any suitable structure that maintains an attached thromboresistant material in a desired position, orientation or range of motion to perform a desired function. Preferably, the support means permits the thromboresistant material to perform a valving function to regulate fluid flow within a body vessel. More preferably, the support means is a support frame attached to one or more thromboresistant valve leaflets. Side views of one particularly preferred implantable valve 100 are shown in FIG. 2A and FIG. 2B, and FIG. 2C shows a perspective view of the same implantable valve 100. The view of FIG. 2B is formed by rotating the view of FIG. 2A 90-degrees into the plane of the page around a central longitudinal axis 101 within the plane of the page. The implantable valve 100 comprises a thromboresistant material 104 attached to a support frame 102. In the implantable valve 100, a first leaflet 140 and a second leaflet 142 formed from flexible thromboresistant material 104 are attached to the support frame 102. The space between the leaflets forms an interior lumen connected with a valve orifice 150 positioned at the proximal end of the two leaflets.

The support frame 102 comprises a plurality of longitudinal struts 110 connecting a first sinusoidal hoop member 120 and a second sinusoidal hoop member 125. The plurality of longitudinal struts 110 comprises a first strut 110a and a second strut 110b, which are labeled in the views of FIG. 2A and FIG. 2B to show the relative orientation of the implantable valve 100 between the two views. Each sinusoidal hoop member 120, 125 comprises a plurality of struts and bends. Adjacent struts within each sinusoidal hoop member 120, 125 are connected by curved support members 130, 132, including a first support member 132a (labeled to show the relative orientation of the implantable valve 100 in FIG. 2A and FIG. 2B). Each leaflet 140, 142 is attached to a longitudinal strut 110 and a strut portion of the second sinusoidal hoop member 120. For example, the first leaflet 140 is attached to the longitudinal struts 110(a) and 110(d). Preferably, the support frame is formed from a self-expanding nickel-titanium alloy, such as Nitinol.

The support frame can have any suitable size. For implantation in a vein, for example, a support frame is preferably expands to diameter of about 2 mm to about 50 mm, more typically between about 8 mm and about 20 mm in diameter. The length of the longitudinal struts 110 can vary, but all struts are preferably substantially the same length. The longitudinal struts 110 are preferably at least about 5% of the total length of the support frame 102, more preferably at least about 10%, 20%, 30%, 40%, 50%, 55%, 60%, 65% or more of the total length of the support frame 102, as measured along the longitudinal axis 101. The length of a longitudinal strut 110 is most preferably about 40% to about 60% of the total length of the support frame 102, as measured along the longitudinal axis 101.

Preferably, a valve leaflet is moveable in response to fluid flow within the frame lumen to regulate fluid flow in a substantially unidirectional manner therethrough. Optionally, the implantable frame can also comprise a thromboresistant material or thromboresistant bioactive agent. Preferably, each leaflet 140, 142 is formed from a biocompatible polyurethane thromboresistant material, most preferably a non-porous polyurethane sold under the tradename THORALON® (Thoratec). The thromboresistant material can be attached to the support frame in any suitable manner, including sutures, heat-sealing, adhesives, tissue welding, weaving, cross-linking or other suitable means for attaching. Referring to the medical device 100 shown in FIG. 2A-FIG. 2C, the first leaflet 140 is a sheet of thromboresistant material attached to the support frame 102 along three edges: a first edge is a distal attachment edge 146 sealably and continuously connected to a portion of the second sinusoidal hoop member 125, and the two adjacent edges are attached to longitudinal struts 110 positioned on opposite sides of the frame 102. Similarly, three sides of the second leaflet 142 are similarly connected to the support frame 102: a distal attachment edge 147 is sealably and continuously connected to a portion of the second sinusoidal hoop member 125, and the two adjacent edges are attached to longitudinal struts 110.

FIG. 2C is a perspective view of the valve 100, which functions to permit fluid to flow in a first direction 101 while substantially preventing fluid flow in a second direction 107. The darkened portion of the frame 102 is positioned behind the first leaflet 140 or the second leaflet 142. The pair of opposable leaflets 140, 142 are attached to the frame 102 on three sides, and each comprise one unattached side 141, 143 that cooperably define a valve orifice. Fluid contact with the leaflets 140, 142 results in the opening and closing of the valve orifice. Preferably, the leaflets 140, 142 are sufficiently flexible to move in response to changes in fluid pressure or direction, and are adapted to effectively regulate fluid in a substantially unidirectional manner. In operation within a body vessel, the implantable valve 100 functions as a one-way valve: fluid flows through the interior lumen 151 in a first direction 106, causing the valve orifice 150 to open, permitting fluid flow through the implantable valve 100. However, when fluid flows in the retrograde (opposite) direction 107, the valve orifice 150 closes as fluid contact moves the first leaflet free edge 141 against the second leaflet free edge 143.

FIG. 2F is an end view of the medical device shown in FIG. 2A-FIG. 2C, showing the first sinusoidal hoop member 120, the valve orifice 150 in the open position, defined by portions of the first leaflet 140 and the second leaflet 142. The proximal leaflet free edge of each leaflet are opposably positioned to define a valve orifice 150. The first leaflet 140 comprises a first leaflet free edge 141; the second leaflet 142 comprises a second leaflet free edge 143. Each leaflet free edge 141, 143 are moveable in response to fluid flow contacting the medical device. The medical device can comprise a valve structure and an expandable support frame configured to provide a sinus region or pocket between a valve leaflet and the farthest radial dimension of the support frame. In the implantable valve 100, as fluid flows in the retrograde direction 107, the fluid fills a first sinus region 152 and an opposably formed second sinus region 154. The first sinus region is formed by the first leaflet 140 on one side, and the body vessel and portions of the support frame 102 on all other sides; similarly, the second sinus region is formed by the second leaflet 142 on one side, and the body vessel and portions of the support frame 102 on all other sides.

FIG. 2D shows a cross-section along line segment A-A′ in FIG. 2A, showing a pair of longitudinal struts 110, including the first longitudinal strut 110a, the first leaflet 140, the second leaflet 142 and a portion of the lumen 151 of the medical device. The thromboresistant material 104 is formed continuously around the longitudinal struts 110. The thromboresistant material can be attached to the longitudinal struts 110 by coating a solution of the thromboresistant material 104 around the implantable frame 102 and allowing the thromboresistant material 104 to dry around portions of the implantable frame 102, for example by spraying the solution of the thromboresistant material 104 onto a mandrel as described below. A tissue adhesion region 144(a) is also shown, which comprises a porous polyurethane material, optionally combined with a fluidized small intestine submucosal material prior to application to the frame.

Similarly, FIG. 2E shows a cross-section along line segment B-B′ in FIG. 2B, showing a pair of longitudinal struts 110, including the first longitudinal strut 110a, the first leaflet 140, the second leaflet 142 and a portion of the lumen 151 of the medical device. The valve leaflets can have a uniform thickness or a thickness that varies at different positions along the valve leaflet. For example, a valve leaflet can be thicker near points of attachment to a support frame, and thinner near a valve orifice region. The tissue adhesion region 144(a) is also shown.

A valve leaflet can have any suitable thickness, and the thickness of a valve leaflet can be uniform or can vary over the surface of the leaflet. The leaflet thickness is preferably calibrated to allow for an adequate flexibility and responsiveness to conditions within a body vessel at a point of implantation. In some embodiments, the thickness of the leaflet can be greater along the perimeter. Thickening the perimeter of valve leaflets can be desirable to promote retention of the valve shape and prevent prolapse or leaflet inversion within a body vessel. Thicker perimeter regions can be formed, for example, by spraying coating additional layers of a thromboresistant material selectively to the perimeter region while masking the central region of the valve leaflet so as to prevent further deposition thereon. Preferably, valve leaflet have a thickness of between about 0.0001 inch and about 0.0030 inch, including thickness of 0.0040, 0.0030, 0.0020, 0.0010, 0.0008, 0.0006, 0.0005, 0.0004, 0.0003, and 0.0002-inch, and more preferably about 0.0030 to about 0.0005 inch thick. The thickness can be measured by any conventional technique, including a conventional micrometer. Preferably, a venous valve leaflet has a variation in thickness of about 20%, more preferably about 10%, or less.

The one or more valve leaflets can be configured and positioned to regulate fluid flow through the implanted medical device. The implantable valve preferably comprises a valve orifice moveable to regulate fluid flow through the valve. The valve orifice can be formed by moveable portions of an implantable frame, by flexible free edges of a flexible material attached to the implantable frame, by a portion of the body vessel, or any combination thereof. Preferred implantable valve structures comprise two or three valve leaflets, although valves can comprise more or fewer leaflets.

The support frame can include structural features, such as barbs, that maintain the support frame in position following implantation in a body vessel. The art provides a wide variety of structural features that are acceptable for use in the support frame, and any suitable structural feature can be used. Furthermore, barbs can also comprise separate members attached to the support frame by suitable attachment means and techniques, such as welding and bonding. For example, referring again to FIGS. 2A-2B, the implantable valve 100 comprises a plurality of barbs 164 positioned to secure the support frame 102 in a body vessel. While the implantable valve 100 comprises two leaflets 140, 142, the implantable valve can also be modified to provide a frame with any suitable number of leaflets attached thereto. For example, embodiments comprising one, three, four, five, six, seven, eight or more leaflets can also be formed by reconfiguring the support frame. For example, support frames comprising additional repeating cell structures can provide implantable valves with three or more leaflets with opposable free edges defining a valve orifice. However, embodiments providing one, two or three leaflets are particularly preferred.

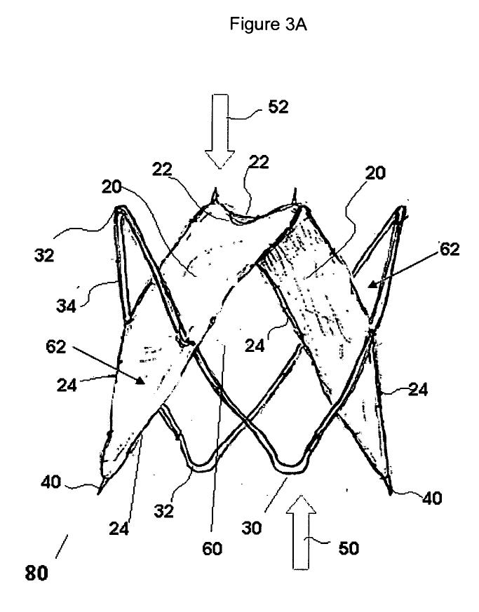

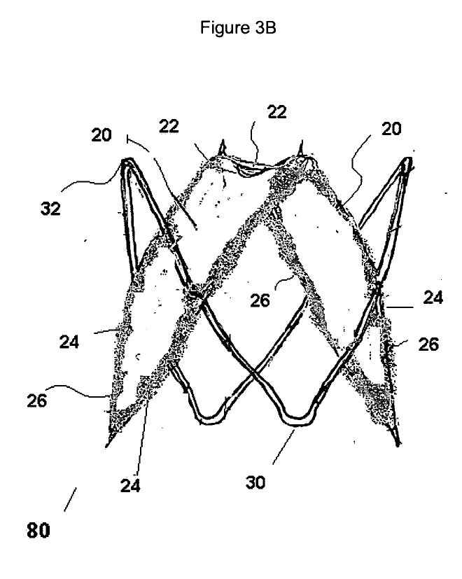

FIG. 3A is a second implantable valve 80 comprising a pair of valve leaflets 20 and a support frame 30. The support frame 30 comprises a plurality of alternating struts 34 and bends 32 arranged in a “zig-zag” pattern and joined into a ring. The valve leaflets 20 each have three edges, including a free edge 22 that is unattached to the support frame 30. The remaining sides 24 of the valve leaflets 20 are attached to the support frame 30. In operation within a body vessel, the implantable valve 80 functions as a one-way valve: fluid flows through an interior lumen 60 defined by the support frame 30 and the valve leaflets 20, in a first direction 50. Movement of fluid in the first direction 50 causes a valve orifice defined by the opposable leaflet free edges 22 to open, permitting fluid flow through the implantable valve 10. However, when fluid flows in the retrograde (opposite) direction 52, the opposable leaflet free edges 22 of the valve orifice close as fluid contact moves the leaflet free edges 22 into contact. Within the body vessel, the valve structure 80 defines a pair of sinus pockets 62, defining pocket between a valve leaflet 20 and the edge of the body vessel (i.e., the farthest radial dimension of the support frame 30). As fluid flows in the retrograde direction 52, the fluid fills the sinus pockets 62, exerting closing pressure on the face of the leaflet 20 toward the longitudinal axis of the lumen 60.

Preferably, the support frame 30 is adapted for intraluminal implantation in a body vessel using a catheter delivery system and is moveable between a compressed configuration for delivery within the catheter delivery system to an expanded configuration upon deployment within the body vessel. The support frame 30 is preferably radially self-expanding, and is shown in a radially expanded configuration. The support frame 30 comprises a self-expanding nickel titanium alloy sold under the tradename NITINOL. Upon compression, self-expanding frames can expand toward their pre-compression geometry. A self-expanding frame can be sized and configured to exert an outward radial force on a body vessel upon implantation, for example to secure the frame in the body vessel or to exert an outward radial force on the body vessel (for example, to perform a “stenting” function). Alternatively, the support frame 30 can be formed from a non-self-expanding material such as a cobalt chromium alloy or stainless steel, and can be balloon expandable. In some embodiments, a support frame can be compressed into a low-profile delivery conformation and then constrained within a delivery system for delivery to a point of treatment in the lumen of a body vessel. At the point of treatment, the support frame can be opened to the radially expanded configuration. The support frame 30 can have any suitable size. For implantation in a vein, for example, a support frame is preferably expands to diameter of about 2 mm to about 50 mm, more typically between about 8 mm and about 20 mm in diameter. The length of the struts 34 can vary, but all struts 34 are preferably substantially the same length. The support frame can include structural features, such as barbs 40, that maintain the support frame in position following implantation in a body vessel. The art provides a wide variety of structural features that are acceptable for use in the support frame, and any suitable structural feature can be used. Furthermore, barbs can also comprise separate members attached to the support frame by suitable attachment means and techniques, such as welding and bonding.

The valve leaflets 20 can be configured in any suitable manner that permits the leaflet to regulate fluid flow across the valve within a body vessel. A valve leaflet 20 can have any suitable thickness, and the thickness of a valve leaflet can be uniform or can vary over the surface of the leaflet. The leaflet thickness is preferably calibrated to allow for an adequate flexibility and responsiveness to conditions within a body vessel at a point of implantation. Substantially uniform coatings can have a thickness variation of less than about 20%, preferably less than about 10%, more preferably less than about 5%, and most preferably less than about 2%. Alternatively, in some embodiments, the thickness of the leaflet can be greater along the perimeter. Thickening the perimeter of valve leaflets can be desirable to promote retention of the valve shape and prevent prolapse or leaflet inversion within a body vessel. Thicker perimeter regions can be formed around one or more sides 24 of the valve leaflet 20, for example, by spraying coating additional layers of a thromboresistant material selectively to the perimeter region while masking the central region of the valve leaflet so as to prevent further deposition thereon. The free edge 22 of a valve leaflet typically has a substantially uniform thickness of up to about 0.005-inch, and preferably between about 0.0001-inch and about 0.003-inch.

The valve leaflets 20 can be formed from any material that is sufficiently flexible to permit movement of the leaflet free to move in response to fluid flow within the frame lumen of the valve to regulate fluid flow in a substantially unidirectional manner therethrough. Preferably, a valve leaflet comprises a flexible biocompatible polyurethane material, most preferably a non-porous polyurethane sold under the tradename THORALON. The thromboresistant material can be attached to the support frame in any suitable manner, including sutures, heat-sealing, adhesives, tissue welding, weaving, cross-linking or other suitable means for attaching.

While the implantable valve 80 comprises two leaflets 20, the implantable valve can also be modified to provide a frame with any suitable number of leaflets attached thereto. For example, embodiments comprising one, three, four, five, six, seven, eight or more leaflets can also be formed by reconfiguring the support frame. For example, support frames comprising additional repeating cell structures can provide implantable valves with three or more leaflets with opposable free edges defining a valve orifice. However, embodiments providing one, two or three leaflets are particularly preferred.

The medical devices of the embodiments described herein may be oriented in any suitable absolute orientation with respect to a body vessel. The recitation of a “first” direction is provided as an example. Any suitable orientation or direction may correspond to a “first” direction. The medical devices of the embodiments described herein may be oriented in any suitable absolute orientation with respect to a body vessel. For example, the first direction can be a radial direction in some embodiments. Also provided are embodiments wherein the medical device comprises a means for orienting the frame within a body lumen. For example, the frame can comprise a marker, such as a radiopaque portion of the frame that would be seen by remote imaging methods including X-ray, ultrasound, Magnetic Resonance Imaging and the like, or by detecting a signal from or corresponding to the marker. In other embodiments, the delivery device can comprise a frame with indicia relating to the orientation of the frame within the body vessel. In other embodiments, indicia can be located, for example, on a portion of a delivery catheter that can be correlated to the location of the frame within a body vessel. The addition of radiopacifiers (i.e., radiopaque materials) to facilitate tracking and positioning of the medical device may be added in any fabrication method or absorbed into or sprayed onto the surface of part or all of the medical device. The degree of radiopacity contrast can be altered by implant content. Radiopacity may be imparted by covalently binding iodine to the polymer monomeric building blocks of the elements of the implant. Common radiopaque materials include barium sulfate, bismuth subcarbonate, and zirconium dioxide. Other radiopaque elements include: cadmium, tungsten, gold, tantalum, bismuth, platinum, iridium, and rhodium. Radiopacity is typically determined by fluoroscope or x-ray film.

In a third embodiment, an implantable valve comprising an adhesion promoting body vessel contact region is provided. The adhesion promoting region of the implantable valve is adapted to promote adhesion of the contact region of the implantable valve to the surface of a body vessel, preferably by promoting the ingrowth of cells and tissue from the body vessel into the contact region of the implanted valve. The adhesion promoting region of the implantable valve can comprise a remodelable material, a porous thromboresistant polyurethane polymer, a tissue growth promoting bioactive agent such as a growth factor, a thromboresistant bioactive agent, or any combination thereof. FIG. 3B shows an implantable valve 80 of FIG. 1A, further comprising an adhesion promoting body vessel contact region 26. The implantable valve is formed from a pair of flexible leaflets 20 comprising a biocompatible polyurethane and attached to a support frame 30. The adhesion promoting body vessel contact region 26 is positioned along the edges 24 of the leaflet that are attached to the support frame 30. The valve leaflets 20 also include opposably positioned free edges 22 forming a valve orifice.

The adhesion promoting region 26 of the implantable valve is adapted to promote adhesion of the contact region of the implantable valve to the surface of a body vessel, preferably by promoting the ingrowth of cells and tissue from the body vessel into the contact region of the implanted valve. The adhesion promoting region 26 of the implantable valve 80 can comprise an extracellular matrix material, a porous thromboresistant polyurethane polymer, a tissue growth promoting bioactive agent such as a growth factor, a thromboresistant bioactive agent, or any combination thereof. Preferred materials for forming an adhesion promoting region include: fibronectin, porous forms of a biocompatible polyurethane, an extracellular matrix material, and combinations thereof. Any implantable device, including a frameless valve and implantable valves comprising a support frame, can comprise one or more adherence promoting region. Each adhesion promoting body vessel contact region 26 comprises a portion of the implantable valve 100 that contacts the interior wall of a body vessel upon implantation therein. The adhesion promoting body vessel contact region 26 can be configured as a coating layer comprising a material selected to promote adhesion of the contact region of the implantable valve to the surface of a body vessel, such as a remodelable extracellular matrix material or a porous biocompatible thromboresistant polymer, such as THORALON. The adhesion promoting body vessel contact region 26 can include a material attached to the support frame 30 and/or to the valve leaflet 20 material. Optionally, a portion of the valve leaflet can be masked during application of the adhesion promoting body vessel contact region 26 material.

Preferably, adhesion promoting body vessel contact region 26 is formed by depositing a porous polyurethane polymer to portions of an implantable valve configured to contact the surface of a body vessel upon implantation to form one or more adhesion promoting regions. Alternatively, a remodelable material such as small intestine submucosa can be attached to portions of the implantable frame 30 by any suitable means, including cross-linking, adhesives, sutures, tissue welding and the like. In other embodiments, a remodelable material is attached to the portions of the implantable frame 30 and a porous biocompatible material or a bioactive agent is applied to or impregnated in the remodelable material. Finally, the adhesion promoting region 26 can also comprise a two component bonding agent such as fibrin glue (e.g., having thrombin and fibrinogen as separate components). To prepare such prostheses, subsequent layers are added after coating the previously-applied layer with a first component of the bonding agent (e.g., thrombin) and coating a layer to be applied with a second component of the bonding agent (e.g., fibrinogen). Thereafter, the layer to be applied is positioned over the previously-applied layer so as to bring the two bonding components into contact, thus causing the curing process to begin. This process can be repeated for any and all additional layers in a laminated construct. Additionally this process can be used to bond the ends of a prosthesis together in vivo. The valve leaflets may also be adhered to the frame using fibrin glue.

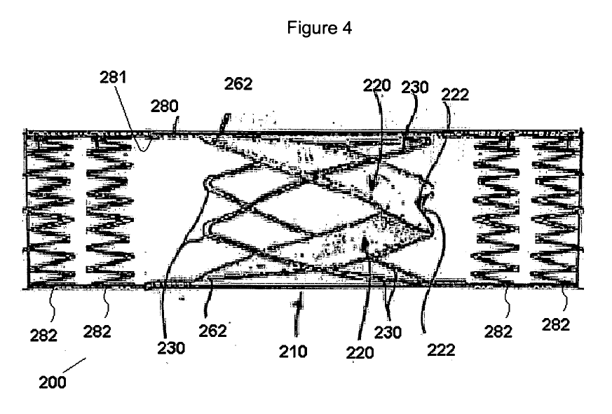

Optionally, a valve may be placed within a sleeve comprising a thromboresistant material. The valve placed within the sleeve may comprise leaflets comprising any suitable material, such as an extracellular matrix material and/or a biocompatible polyurethane. FIG. 4 shows an implantable valve 200 comprising an outer sleeve 280 enclosing an implantable valve 210 that is substantially similar to the valve 80 of FIG. 3A. The outer sleeve 280 can be configured as a tube enclosing a valve means. The valve means can be an implantable valve 210 comprising a one or more valve leaflets 220 support frame 230. The leaflet free edges 222 can form a valve orifice moveable to permit fluid flow in a first direction 250, while closing to prevent fluid flow in a retrograde direction 252. When the valve orifice is closed, fluid flowing in the retrograde direction 252 can collect in sinus pockets 262 formed between the inner surface of the outer sleeve 280 and the outer surface of the valve leaflets 220. The outer sleeve 280 is preferably formed from one or more layers of a biocompatible polyurethane. Optionally, the outer sleeve 280 can be supported by a sleeve support frame having any suitable configuration to provide a desired shape and stability to the outer sleeve 280. For example, the sleeve support frame can include a plurality of sinusoidal hoop members 282 formed from a self-expanding biocompatible metal or metal alloy. The hoop members 282 can be positioned at either end of the outer sleeve 280 and can exert a force in an outward radial direction to secure the ends of the outer sleeve 280 against the inner wall of a body vessel. The outer sleeve 280 is preferably formed by casting a biocompatible polyurethane material on the inner wall of a cylindrical mold, independent of and prior to placement of the valve means 210 within the lumen of the outer sleeve 280. For example, the outer sleeve 280 can be formed by placing the hoop members 282 within a suitable cylindrical mold, introducing a suitable amount of the biocompatible polyurethane as a solution in a suitable volatile organic solvent, and slowly rotating the cylindrical mold around the longitudinal axis to evaporate the solvent. After removal of the solvent, an outer sleeve structure 280 enclosing the hoop members 282 can be removed from the cylindrical mold. The valve 210 can then be placed within the lumen of the outer sleeve structure 280, and secured therein. Optionally, the outer sleeve structure 280 can be configured as a stent graft, such as the composite stent graft disclosed by Hartley in U.S. Patent Application Publication No. US 2005/0131519A 1, filed Oct. 12, 2004 and incorporated herein by reference in its entirety.

Biocompatible Polyurethane Materials

Preferably, an implantable medical device for placement within a body passage comprises one or more thromboresistant materials. The thromboresistant material is preferably a biocompatible polyurethane material optionally including a thromboresistant bioactive agent, an extracellular matrix material comprising a thromboresistant bioactive agent, or a combination thereof. Preferably, the thromboresistant material is a biocompatible polyurethane material comprising a surface modifying agent, as described herein.

The thromboresistant material, as disclosed herein, can be selected from a variety of materials, but preferably comprises a biocompatible polyurethane material. One particularly preferred biocompatible polyurethane is THORALON (THORATEC, Pleasanton, Calif.), described in U.S. Pat. Application Publication No. 2002/0065552 A1 and U.S. Pat. No. 4,675,361, both of which are incorporated herein by reference. The biocompatible polyurethane material sold under the tradename THORALON is a polyurethane base polymer (referred to as BPS-215) blended with a siloxane containing surface modifying additive (referred to as SMA-300). The concentration of the surface modifying additive may be in the range of 0.5% to 5% by weight of the base polymer.

THORALON has been used in certain vascular applications and is characterized by thromboresistance, high tensile strength, low water absorption, low critical surface tension, and good flex life. THORALON is believed to be biostable and to be useful in vivo in long term blood contacting applications requiring biostability and leak resistance. Because of its flexibility, THORALON is useful in larger vessels, such as the abdominal aorta, where elasticity and compliance is beneficial.

The SMA-300 component (THORATEC) is a polyurethane comprising polydimethylsiloxane as a soft segment and the reaction product of diphenylmethane diisocyanate (MDI) and 1,4-butanediol as a hard segment. A process for synthesizing SMA-300 is described, for example, in U.S. Pat. Nos. 4,861,830 and 4,675,361, which are incorporated herein by reference.

The BPS-215 component (THORATEC) is a segmented polyetherurethane urea containing a soft segment and a hard segment. The soft segment is made of polytetramethylene oxide (PTMO), and the hard segment is made from the reaction of 4,4′-diphenylmethane diisocyanate (MDI) and ethylene diamine (ED).

THORALON can be formed as non-porous material or as a porous material with varying degrees and sizes of pores, as described below. Implantable medical devices can comprise one or both forms of THORALON. The thromboresistant material preferably comprises the non-porous form of THORALON. The porous forms of THORALON can also be used as a thromboresistant material, but are preferably employed as an adhesion promoting region. For example, valve leaflets preferably comprise non-porous THORALON as a thromboresistant material, while adhesion promoting body vessel contact region on the outside of a prosthetic valve preferably comprise porous THORALON as an adhesion promoting material.

Porous THORALON can be formed by mixing the polyetherurethane urea (BPS-215), the surface modifying additive (SMA-300) and a particulate substance in a solvent. The particulate may be any of a variety of different particulates or pore forming agents, including inorganic salts. Preferably the particulate is insoluble in the solvent. The solvent may include dimethyl formamide (DMF), tetrahydrofuran (THF), dimethyacetamide (DMAC), or dimethyl sulfoxide (DMSO), or mixtures thereof. The composition can contain from about less than 1 wt % to about 40 wt % polymer, and different levels of polymer within the range can be used to fine tune the viscosity needed for a given process. The composition can contain less than 5 wt % polymer for some spray application embodiments, such as 0.1-5.0 wt %. For dipping application methods, compositions desirably comprise about 5 to about 25 wt %. The particulates can be mixed into the composition. For example, the mixing can be performed with a spinning blade mixer for about an hour under ambient pressure and in a temperature range of about 18° C. to about 27° C. The entire composition can be cast as a sheet, or coated onto an article such as a mandrel or a mold. In one example, the composition can be dried to remove the solvent, and then the dried material can be soaked in distilled water to dissolve the particulates and leave pores in the material. In another example, the composition can be coagulated in a bath of distilled water. Since the polymer is insoluble in the water, it will rapidly solidify, trapping some or all of the particulates. The particulates can then dissolve from the polymer, leaving pores in the material. It may be desirable to use warm water for the extraction, for example water at a temperature of about 60° C. The resulting pore diameter can also be substantially equal to the diameter of the salt grains. The resulting void-to-volume ratio, as defined above, can be substantially equal to the ratio of salt volume to the volume of the polymer plus the salt. Formation of porous THORALON is described, for example, in U.S. Pat. Application Publication Nos. 2003/0114917 A1 and 2003/0149471 A1, both of which are incorporated herein by reference.

A variety of other biocompatible polyurethanes/polycarbamates and urea linkages (hereinafter “—C(O)N or CON-type polymers”) may also be employed. These include CON type polymers that preferably include a soft segment and a hard segment. The segments can be combined as copolymers or as blends. For example, CON type polymers with soft segments such as PTMO, polyethylene oxide, polypropylene oxide, polycarbonate, polyolefin, polysiloxane (i.e. polydimethylsiloxane), and other polyether soft segments made from higher homologous series of diols may be used. Mixtures of any of the soft segments may also be used. The soft segments also may have either alcohol end groups or amine end groups. The molecular weight of the soft segments may vary from about 500 to about 5,000 g/mole.

Preferably, the hard segment is formed from a diisocyanate and diamine. The diisocyanate may be represented by the formula OCN—R—NCO, where —R— may be aliphatic, aromatic, cycloaliphatic or a mixture of aliphatic and aromatic moieties. Examples of diisocyanates include MDI, tetramethylene diisocyanate, hexamethylene diisocyanate, trimethyhexamethylene diisocyanate, tetramethylxylylene diisocyanate, 4,4′-dicyclohexylmethane diisocyanate, dimer acid diisocyanate, isophorone diisocyanate, metaxylene diisocyanate, diethylbenzene diisocyanate, decamethylene 1,10 diisocyanate, cyclohexylene 1,2-diisocyanate, 2,4-toluene diisocyanate, 2,6-toluene diisocyanate, xylene diisocyanate, m-phenylene diisocyanate, hexahydrotolylene diisocyanate (and isomers), naphthylene-1,5-diisocyanate, 1-methoxyphenyl 2,4-diisocyanate, 4,4′-biphenylene diisocyanate, 3,3′-dimethoxy-4,4′-biphenyl diisocyanate and mixtures thereof.

The diamine used as a component of the hard segment includes aliphatic amines, aromatic amines and amines containing both aliphatic and aromatic moieties. For example, diamines include ethylene diamine, propane diamines, butanediamines, hexanediamines, pentane diamines, heptane diamines, octane diamines, m-xylylene diamine, 1,4-cyclohexane diamine, 2-methypentamethylene diamine, 4,4′-methylene dianiline, and mixtures thereof. The amines may also contain oxygen and/or halogen atoms in their structures.

Other applicable biocompatible polyurethanes include those using a polyol as a component of the hard segment. Polyols may be aliphatic, aromatic, cycloaliphatic or may contain a mixture of aliphatic and aromatic moieties. For example, the polyol may be ethylene glycol, diethylene glycol, triethylene glycol, 1,4-butanediol, 1,6-hexanediol, 1,8-octanediol, propylene glycols, 2,3-butylene glycol, dipropylene glycol, dibutylene glycol, glycerol, or mixtures thereof.

Biocompatible CON type polymers modified with cationic, anionic and aliphatic side chains may also be used. See, for example, U.S. Pat. No. 5,017,664. Other biocompatible CON type polymers include: segmented polyurethanes, such as BIOSPAN; polycarbonate urethanes, such as BIONATE; and polyetherurethanes, such as ELASTHANE; (all available from POLYMER TECHNOLOGY GROUP, Berkeley, Calif.). Other biocompatible CON type polymers can include polyurethanes having siloxane segments, also referred to as a siloxane-polyurethane. Examples of polyurethanes containing siloxane segments include polyether siloxane-polyurethanes, polycarbonate siloxane-polyurethanes, and siloxane-polyurethane ureas. Specifically, examples of siloxane-polyurethane include polymers such as ELAST-EON 2 and ELAST-EON 3 (AORTECH BIOMATERIALS, Victoria, Australia); polytetramethyleneoxide (PTMO) and polydimethylsiloxane (PDMS) polyether-based aromatic siloxane-polyurethanes such as PURSIL-10, -20, and -40 TSPU; PTMO and PDMS polyether-based aliphatic siloxane-polyurethanes such as PURSIL AL-5 and AL-10 TSPU; aliphatic, hydroxy-terminated polycarbonate and PDMS polycarbonate-based siloxane-polyurethanes such as CARBOSIL-10, -20, and -40 TSPU (all available from POLYMER TECHNOLOGY GROUP). The PURSIL, PURSIL-AL, and CARBOSIL polymers are thermoplastic elastomer urethane copolymers containing siloxane in the soft segment, and the percent siloxane in the copolymer is referred to in the grade name. For example, PURSIL-10 contains 10% siloxane. These polymers are synthesized through a multi-step bulk synthesis in which PDMS is incorporated into the polymer soft segment with PTMO (PURSIL) or an aliphatic hydroxy-terminated polycarbonate (CARBOSIL). The hard segment consists of the reaction product of an aromatic diisocyanate, MDI, with a low molecular weight glycol chain extender. In the case of PURSIL-AL the hard segment is synthesized from an aliphatic diisocyanate. The polymer chains are then terminated with a siloxane or other surface modifying end group. Siloxane-polyurethanes typically have a relatively low glass transition temperature, which provides for polymeric materials having increased flexibility relative to many conventional materials. In addition, the siloxane-polyurethane can exhibit high hydrolytic and oxidative stability, including improved resistance to environmental stress cracking. Examples of siloxane-polyurethanes are disclosed in U.S. Pat. Application Publication No. 2002/0187288 A1, which is incorporated herein by reference.

In addition, any of these biocompatible CON type polymers may be end-capped with surface active end groups, such as, for example, polydimethylsiloxane, fluoropolymers, polyolefin, polyethylene oxide, or other suitable groups. See, for example the surface active end groups disclosed in U.S. Pat. No. 5,589,563, which is incorporated herein by reference.

Growth Factors

In a fourth embodiment, the medical device comprises a surface formed from a biocompatible polyurethane material comprising a growth factor and optionally further comprising a remodelable material. The biocompatible polyurethane or remodelable material preferably comprises one or more growth factors. Without being bound to theory, it is believed that the presence of one or more growth factors may promote deposition of endothelial cells over the surface of a medical device within a body vessel, resulting in a lower likelihood of thrombus formation. The growth factor agent is preferably incorporated within the biocompatible polyurethane by any suitable method. In one aspect, the growth factor or is incorporated in an implantable valve by soaking a valve leaflet comprising a porous biocompatible polyurethane portion in a solution, such as phosphate buffered saline, comprising the desired growth factor. In another aspect, the valve can comprise a remodelable material comprising a growth factor.

Non-limiting examples of growth factors include: fibroblast growth factors (FGF) (e.g., FGF1, FGF2, FGF3, FGF4, FGF5, FGF6, FGF7, FGF8, FGF9, and FGF10), epidermal growth factor, keratinocyte growth factor, vascular endothelial cell growth factors (VEGF) (e.g., VEGF A, B, C, D, and E), placenta growth factor (PIGF), platelet-derived growth factor (PDGF), epidermal growth factor (EGF), interferons (IFN) (e.g., IFN-alpha, beta, or gamma), transforming growth factors (TGF) (e.g., TGF.alpha or beta), tumor necrosis factor-.alpha, an interleukin (IL) (e.g., IL-1-IL-18), Osterix (See, e.g., Tai G. et al., “Differentiation of osteoblasts from murine embryonic stem cells by overexpression of the transcriptional factor osterix,” Tissue Eng. 2004 September-October; 10(9-10): 1456-66, incorporated herein by reference in its entirety), Hedgehogs (e.g., sonic or desert) (See, e.g., Adolphe C. et al., “An in vivo comparative study of sonic, desert and Indian hedgehog reveals that hedgehog pathway activity regulates epidermal stem cell homeostasis,” Development. 2004 October;131(20):5009-19. Epub 2004 Sep 15, incorporated herein by reference in its entirety), bone morphogenic proteins, basic fibroblast growth factor (bFGF), parathyroid hormone, calcitonin prostaglandins, ascorbic acid, and hepatocyte growth factor.

In one embodiment, a valve leaflet comprises at least one fibroblast growth factor, preferably basic fibroblast growth factor FGF-2. In some embodiments, a valve leaflet comprises at least one Transforming Growth Factor, preferably TGF-beta. In one preferred embodiment, a valve leaflet comprises both FGF-2 and TGF-beta. Other preferred growth factors include one or more types of EGFs (epidermal growth factors), PDGFs (platelet derived growth factors), and VEGFs (vascular endothelial growth factor). See, e.g., Sachiyo Ogawa, et al., “A Novel Type of Vascular Endothelial Growth Factor, VEGF-E (NZ-7 VEGF), Preferentially Utilizes KDR/Flk-1 Receptor and Carries a Potent Mitotic Activity without Heparin-binding Domain,” J Biol Chem, Vol. 273, Issue 47, 31273-31282, Nov. 20, 1998, incorporated herein by reference). Other examples of growth factors include: Brain-derived Neurotrophic Factor, Epidermal Growth Factor, Fibroblast Growth Factor, Endothelial cell growth supplement, Granulocyte-Macrophage Colony-Stimulating Factor, Hepatocyte Growth Factor, Insulin-like Growth Factor, Interleukins, Leukemia Inhibitory Factor, Nerve Growth Factor, Platelet-Derived Growth Factor, Transforming Growth Factor, Tumor Necrosis Factor, and Vascular Endothelial Growth Factor.

The following non-limiting examples of other references relating to growth factors and ECM materials are incorporated herein by reference: Zheng B, Clemmons D R, “Methods for preparing extracellular matrix and quantifying insulin-like growth factor-binding protein binding to the ECM,” Methods Mol. Biol. 2000;139:221-30; Rosso F, et. al., “From cell-ECM interactions to tissue engineering,” J Cell Physiol. 2004 May; 199(2):174-80; Pollak M N, “Insulin-like growth factors and neoplasia,” Novartis Found Symp. 2004;262:84-98; discussion 98-107, 265-8; Liu X, et al., “Synergetic effect of interleukin-4 and transforming growth factor-beta1 on type I collagen gel contraction and degradation by HFL-1 cells: implication in tissue remodeling,” Chest. 2003 March;123(3 Suppl):427S-8S and Shukla A, et al., “Perspective article: transforming growth factor-beta: crossroad of glucocorticoid and bleomycin regulation of collagen synthesis in lung fibroblasts,” Wound Repair Regen. 1999 May-June;7(3): 133-40.

More preferably, a valve leaflet comprises a porous biocompatible polyurethane that is soaked in a solution comprising at least about 100 ng of FGF-2 per mL of solution. FGF-2 is a pluripotent mitogen believed to be capable of stimulating migration and proliferation of a variety of cell types including fibroblasts, macrophages, smooth muscle and endothelial cells. In addition to these mitogenic properties, FGF-2 is believed to stimulate endothelial production of various proteases, including plasminogen activator and matrix metalloproteinases, induce significant vasodilation through stimulation of nitric oxide release and promote chemotaxis. FGF-2 binds avidly (K_{d}=10⁻⁹ M) to endothelial cell surface heparin sulfates. This interaction serves to prolong effective tissue half-life of the FGF-2 protein, facilitates its binding to its high-affinity receptors and plays a key role in stimulation of cell proliferation and migration. FGF-2 also possesses a plethora of other biological effects such as the ability to stimulate NO release, to synthesize various proteases, including plasminogen activator and matrix metalloproteinases, and to induce chemotaxis. Homozygous deletion of the bFGF gene is associated with decreased vascular smooth muscle contractility, low blood pressure and thrombocytosis.

Preferably, a valve leaflet comprises two or more growth factors that synergistically interact to promote remodeling of the composition after implantation. Any combination of two or more synergistic growth factors may be used. For example, one or more growth factors can be added to an ECM material to form a composition comprising two or more synergistic growth factors. Preferably, in some embodiments, FGF-2 and VEGF growth factors are combined in a composition to synergistically promote remodeling of the implanted composition. A combination of FGF-2 and VEGF is believed to be far more potent than FGF-2 alone in inducing angiogenesis in vitro and in vivo. Furthermore, FGF-2 induces VEGF expression in smooth muscle and endothelial cells. The synergistic relationship between FGF-2 and VEGF is documented in the literature, for example in the following references which are incorporated herein in their entirety: Bootle-Wilbraham C A, et al., “Fibrin fragment E stimulates the proliferation, migration and differentiation of human microvascular endothelial cells in vitro,” Angiogenesis. 2001;4(4):269-75; Nico B, et al., “In vivo absence of synergism between fibroblast growth factor-2 and vascular endothelial growth factor,” J Hematother Stem Cell Res. 2001 December;10(6):905-12; and Hata Y, et al., “Basic fibroblast growth factor induces expression of VEGF receptor KDR through a protein kinase C and p44/p42 mitogen-activated protein kinase-dependent pathway,” Diabetes. 1999 May;48(5):1145-55.

Vascular endothelial growth factor (VEGF) is a potent and specific mitogen for vascular endothelial cells that is capable of stimulating angiogenesis during embryonic development and tumor formation. The VEGF family of structurally related growth factors has five mammalian members, VEGF, VEGF-B, VEGF-C, VEGF-D, and placenta growth factor (PIGF), all encoded by separate genes. Stacker, S. A. and Achen, M. G. “The vascular endothelial growth factor (VEGF) family: signaling for vascular development.” Growth Factors 17: 1-11 (1999).

A valve leaflet material can be tested for growth factors using any suitable assay identified by one in the art to provide the desired level of sensitivity. In some embodiments, growth factors can be identified using an in vitro assay. Various assays for growth factors are known in the art to identify the presence of growth factors and quantify the concentration of a growth factor. For example, Human FGF Basic ELISA assay can be used to identify certain growth factors. Other examples of growth factor assays are disclosed in U.S. Pat. No. 6,375,989 to Badylak et al., incorporated herein by reference, which discloses in vitro assays using antibodies to identify FGF-2 and TGF-beta in submucosal ECM material. A preferred method for detection of FGF-2 in a composition is the QUANTIKINE HS ® Human FGF basic Immunoassay. The QUANTIKINE HS ® FGF basic Immunoassay kit is a 6.5 hour solid phase ELISA designed to measure FGF basic levels in serum, plasma, and urine. The QUANTIKINE HS ® FGF basic Immunoassay contains E. coli-expressed recombinant human FGF basic and antibodies raised against the recombinant factor. It has been shown to quantitate recombinant human FGF basic accurately.

Remodelable Materials

In another aspect, the fourth embodiment provides valve leaflets comprising a remodelable material optionally combined with or in contact with a thromboresistant bioactive agent. For example, the remodelable material can be attached to an edge 24 of the valve 80, or to the surface of the valve leaflet 20, in FIG. 3A by any suitable means, including attachment to a portion of a support frame using stitching through the thromboresistant material of a leaflet 20 and around a portion of the support frame 30, adhesives, tissue welding or cross linking to directly join the remodelable material to the frame. The remodelable material can also form a second layer laminated to a biocompatible polyurethane layer. Alternatively, a remodelable material can be positioned between two layers of biocompatible polyurethane material to provide a three-layer leaflet structure. Preferably, a biocompatible polyurethane layer positioned over the remodelable material is sufficiently porous to permit adhesion of endothelial cells to the remodelable material within the pores.

A “remodelable material,” as further discussed below, is any material or combination of materials that can undergo biological processes of remodeling when placed in communication with a living tissue, such that the remodelable material is transformed into material that is substantially similar to said living tissue in cellular composition. Unless otherwise specified herein, a “remodelable material” includes a single layer material, or a multiple layers of one or more materials that together undergo remodeling when placed in communication with living tissue. Preferably, a remodelable material undergoes remodeling by tissue and cells from the body vessel upon contact for 90 days or less with living tissue of the type present at an intended site of implantation, such as the interior of a body vessel.

Optionally, polyurethane can be embedded in an extracellular matrix material. The embedded polyurethane can be introduced in any suitable physical form, including sheets, beads or threads. In one preferred composite material, a sheet of small intestine submucosa and a sheet of polyurethane are joined to form a laminate comprising at least two layers. The layers of the composite material can be joined in any suitable manner, including cross linking or heat pressing.

Upon implantation, remodelable materials, such as submucosal tissue, undergo remodeling and induce the growth of endogenous tissues upon implantation into a host. One example of a remodeling process is the migration of cells into the remodelable material. Migration of cells into the remodelable material can occur in various ways, including physical contact with living tissue, or recruitment of cells from tissue at a remote location that are carried in a fluid flow to the remodelable material. In some embodiments, the remodelable material can provide an acellular scaffold or matrix that can be populated by cells. The migration of cells into the remodelable material can impart new structure and function to the remodelable material. In some embodiments, the remodelable material itself can be absorbed by biological processes. In some embodiments, fully remodeled material can be transformed into the living tissue it is in contact with through cellular migration from the tissue into the remodelable material, or provide the structural framework for tissue. Non-limiting examples of remodelable materials, their preparation and use are also discussed herein.

The remodelable material is preferably a reconstituted or naturally-derived collagenous materials. Such materials can promote cellular invasion and ingrowth. Suitable bioremodelable materials can be provided by collagenous extracellular matrix materials (ECMs) possessing biotropic properties, including in certain forms angiogenic collagenous extracellular matrix materials. For example, suitable collagenous materials include ECMs such as submucosa, renal capsule membrane, dermal collagen, dura mater, pericardium, fascia lata, serosa, peritoneum or basement membrane layers, including liver basement membrane. Suitable submucosa materials for these purposes include, for instance, intestinal submucosa, including small intestinal submucosa, stomach submucosa, urinary bladder submucosa, and uterine submucosa.

As prepared, the submucosa material and any other ECM used may optionally retain growth factors or other bioactive components native to the source tissue. For example, the submucosa or other ECM may include one or more growth factors such as basic fibroblast growth factor (FGF-2), transforming growth factor beta (TGF-beta), epidermal growth factor (EGF), and/or platelet derived growth factor (PDGF). As well, submucosa or other ECM used in the invention may include other biological materials such as heparin, heparin sulfate, hyaluronic acid, fibronectin and the like. Thus, generally speaking, the submucosa or other ECM material may include a bioactive component that induces, directly or indirectly, a cellular response such as a change in cell morphology, proliferation, growth, protein or gene expression.

Submucosa or other ECM materials of the present invention can be derived from any suitable organ or other tissue source, usually sources containing connective tissues. The ECM materials processed for use in the invention will typically include abundant collagen, most commonly being constituted at least about 80% by weight collagen on a dry weight basis. Such naturally-derived ECM materials will for the most part include collagen fibers that are non-randomly oriented, for instance occurring as generally uniaxial or multi-axial but regularly oriented fibers. When processed to retain native bioactive factors, the ECM material can retain these factors interspersed as solids between, upon and/or within the collagen fibers. Particularly desirable naturally-derived ECM materials for use in the invention will include significant amounts of such interspersed, non-collagenous solids that are readily ascertainable under light microscopic examination with specific staining. Such non-collagenous solids can constitute a significant percentage of the dry weight of the ECM material in certain inventive embodiments, for example at least about 1%, at least about 3%, and at least about 5% by weight in various embodiments of the invention.

The submucosa or other ECM material used in the present invention may also exhibit an angiogenic character and thus be effective to induce angiogenesis in a host engrafted with the material. In this regard, angiogenesis is the process through which the body makes new blood vessels to generate increased blood supply to tissues. Thus, angiogenic materials, when contacted with host tissues, promote or encourage the infiltration of new blood vessels. Methods for measuring in vivo angiogenesis in response to biomaterial implantation have recently been developed. For example, one such method uses a subcutaneous implant model to determine the angiogenic character of a material. See, C. Heeschen et al., Nature Medicine 7 (2001), No. 7, 833-839. When combined with a fluorescence microangiography technique, this model can provide both quantitative and qualitative measures of angiogenesis into biomaterials. C. Johnson et al., Circulation Research 94 (2004), No. 2, 262-268.

Further, in addition or as an alternative to the inclusion of native bioactive components, non-native bioactive components such as those synthetically produced by recombinant technology or other methods, may be incorporated into the submucosa or other ECM tissue. These non-native bioactive components may be naturally-derived or recombinantly produced proteins that correspond to those natively occurring in the ECM tissue, but perhaps of a different species (e.g. human proteins applied to collagenous ECMs from other animals, such as pigs). The non-native bioactive components may also be drug substances. Illustrative drug substances that may be incorporated into and/or onto the ECM materials used in the invention include, for example, antibiotics or thrombus-promoting substances such as blood clotting factors, e.g. thrombin, fibrinogen, and the like. These substances may be applied to the ECM material as a premanufactured step, immediately prior to the procedure (e.g. by soaking the material in a solution containing a suitable antibiotic such as cefazolin), or during or after engraftment of the material in the patient.

Submucosa or other ECM tissue used in the invention is preferably highly purified, for example, as described in U.S. Pat. No. 6,206,931 to Cook et al. Thus, preferred ECM material will exhibit an endotoxin level of less than about 12 endotoxin units (EU) per gram, more preferably less than about 5 EU per gram, and most preferably less than about 1 EU per gram. As additional preferences, the submucosa or other ECM material may have a bioburden of less than about 1 colony forming units (CFU) per gram, more preferably less than about 0.5 CFU per gram. Fungus levels are desirably similarly low, for example less than about 1 CFU per gram, more preferably less than about 0.5 CFU per gram. Nucleic acid levels are preferably less than about 5 μg/mg, more preferably less than about 2 μg/mg, and virus levels are preferably less than about 50 plaque forming units (PFU) per gram, more preferably less than about 5 PFU per gram. These and additional properties of submucosa or other ECM tissue taught in U.S. Pat. No. 6,206,931 may be characteristic of the submucosa tissue used in the present invention.

A remodelable material may also comprise a bio-compatible material such as Dacron, expanded polytetrafluoroethylene (ePTFE) or other synthetic bio-compatible material. In one embodiment, the remodelable material comprises at least two ECM materials derived from different sources in the same layer, or in different layers. In another embodiment, the remodelable material comprises an ECM material and an elastin material. In yet another embodiment, the remodelable material is a woven material comprising strands of an ECM material woven with another ECM material or a structural reinforcing material such as ePTFE.

Valve leaflets can comprise a laminate of sheets of remodelable material crosslinked to bond multiple sheets to one another. Thus, additional crosslinking may be added to individual submucosa layers prior to bonding to one another, during bonding to one another, and/or after bonding to one another.

Valve leaflets can comprise a first layer formed from a biocompatible polyurethane attached to a remodelable material. In another aspect, the first embodiment provides valve leaflets comprising a first layer formed from a sheet of remodelable material in contact with a biocompatible polyurethane. A biocompatible polyurethane material can comprise a polyurethane polymer cross-linked to an ECM such as small intestine submucosa. Cross linking of these two materials can be accomplished by reacting the ester functionality of SIS with a crosslinking agent containing an oxygen or nitrogen to form an ester or amide bond, respectively. Polyurethane ureas can be cross-linked by reaction of the urea functionality with an oxygen or nitrogen functionality to form a urea bond or urethane bond. Polyamines, polyalcohols or amino alcohols are suitable cross-linking agent to cross-link polyurethane ureas and SIS. Alternatively, an epoxy amine or epoxy alcohol could be used to cross-link a polyurethane and SIS. In this case the amine or alcohol functionality of the cross-linking agent would form an ester or amide bond with the SIS material, and the epoxy functionality of the crosslinking agent would alkylate the urea functionality.

A biocompatible polyurethane can be a composite material comprising a cross-linked ECM material and/or an ECM material cross linked to a polyurethane polymer, for example to strengthen the material. Cross-linking can be performed, for example, to mechanically stabilize the ECM material. Cross-linked material generally refers to material that is completely cross-linked in the sense that further contact with a cross-linking agent does not further change measurable mechanical properties of the material. However, total (100%) cross-linking is not always needed to achieve many desired mechanical properties. Cross-linking of the material preferably involves a chemical cross-linking agent with a plurality of functional groups that bond to the material 30 to form a chemically cross-linked material 30. The chemical cross-linking is preferably performed until a cross-linking agent has permeated the material of the cross-linking region and reacted with the accessible binding sites of the material.

Cross-link bonds can be formed in any suitable manner that provides attachment of a material to an implantable frame, including formation of cross-link chemical bonds between two surfaces of the material and/or formation of a cross-link bond between the frame and a portion of material. For example, cross-linking can be introduced by chemical treatment of the frame and/or material, such as glycolylation. The material can be subjected to a form of energy to introduce cross-linking. For example, energy treatment suitable for use in the invention includes exposing the material to ultraviolet light, heat, or both. In general, the material for use in the medical device and material for leaflet formation can be processed prior to cross-linking the material. For example, the material can undergo cutting and trimming, sterilizing, and associating the material with one or more desirable compositions, such as anticalcification agents and growth factors, and the like. After any preliminary processing and or storage is completed, the material can be cross-linked. Following cross-linking of the material, the material can be further processed, which can involve additional chemical and or mechanical manipulation of the material as well as processing the material into the desired medical device. Other cross-linking agents can be used to form cross-linking regions, such as epoxides, epoxyamines, diimides and other difunctional polyfunctional aldehydes. In particular, aldehyde functional groups are highly reactive with amine groups in proteins, such as collagen. Epoxyamines are molecules that generally include both an amine moiety (e.g. a primary, secondary, tertiary, or quaternary amine) and an epoxide moiety. The epoxyamine compound can be a monoepoxyamine compound and or a polyepoxyamine compound. In some embodiments, the epoxyamine compound is a polyepoxyamine compound having at least two epoxide moieties and possibly three or more epoxide moieties. In some embodiments, the polyepoxyamine compound is triglycidylamine (TGA). The use of cross-linking agents form corresponding adducts, such as glutaraldehyde adducts and epoxyamine adducts, of the cross-linking agent with the material that have an identifiable chemical structures.

Alternatively, ECM and/or polyurethane materials may be cross-linked using radical reactions. A radical is generated in the material to be cross-linked using a free radical generator, such as an organic peroxide of which many are known and commercially available, such as dicumyl peroxide, benzoyl peroxide, and the like. In this case the crosslinking agent is a multifunctional monomer capable of crosslinking the particular polymer when initiated by the free radical generator or irradiation. Typically, the crosslinking agent contains at least two ethylenic double bonds, which may be present, for example, in allyl, methallyl, propargyl or vinyl groups.

The remodelable material and a biocompatible polyurethane material can be intimately mixed by forming a fluidized remodelable material that can be combined with a solution of the polyurethane, which can then be dried into a composite sheet to form remodelable polyurethane material having a desired thickness. The fluidized remodelable compositions are prepared as solutions or suspensions of an extracellular matrix material (ECM) by comminuting and/or digesting the ECM with a protease, such as trypsin or pepsin, for a period of time sufficient to solubilize said tissue and form a substantially homogeneous solution. The ECM starting material can be comminuted by any suitable method (e.g., tearing, cutting, grinding, shearing and the like). Grinding the ECM in a frozen or freeze-dried state is preferred, although a suspension of pieces the ECM can also be comminuted in a high speed (high shear) blender with dewatering, if necessary, by centrifuging and decanting excess water. The comminuted ECM can be dried to form an ECM powder. Thereafter, the ECM can be hydrated, by combining with water or buffered saline and optionally other pharmaceutically acceptable excipients to form a fluidized ECM composition. Optionally, the fluidized material may be subjected to proteolytic digestion to form a substantially homogeneous solution. In one embodiment, the ECM powder is digested with 1 mg/ml of pepsin (Sigma Chemical Co., St. Louis, Mo.) in 0.1 M acetic acid, adjusted to pH 2.5 with HCl, over a 48 hour period at room temperature. The reaction medium is neutralized with sodium hydroxide to inactivate the peptic activity. The solubilized ECM may then be concentrated by salt precipitation of the solution and separated for further purification and/or freeze drying to form a protease solubilized intestinal submucosa in powder form. The viscosity of fluidized ECM compositions can be manipulated by controlling the concentration of the ECM component and the degree of hydration. The viscosity can be adjusted to a range of about 2 to about 300,000 cps at 25.degree. C. Higher viscosity formulations, for example, gels, can be prepared from the SIS digest solutions by adjusting the pH of such solutions to about 6.0 to about 7.0. Additional details pertaining to the preparation of a fluidized ECM remodelable material are found in U.S. Pat. No. 5,275,826, filed Nov. 13, 1993 (Badylak et al.), incorporated herein by reference. One or more polyurethane materials, such as powders, microparticles, nanoparticles, or beads or colloidal suspensions thereof, are preferably mixed with the fluidized ECM material described above. The mixture can be dried into a sheet having a desired thickness to form a valve leaflet.

Alternatively, the polyurethane and remodelable materials can be pressed into one or more sheets to form a composite material for a valve leaflet. For example, polyurethane sheets can be placed between two parallel sheets of small intestine submucosa, which are then pressed together and dried in any manner effective to join the two sheets to form a composite material. For example, the two sheets of small intestine submucosa can be tensionably compressed between two heated nip rollers to seal the materials between the sheets.

Support Frames

An implantable medical device can comprise a support means for providing structural support to the thromboresistant material. The support means can be formed from any suitable structure that maintains an attached thromboresistant material in a desired position, orientation or range of motion to perform a desired function. Preferably, the support means is a radially expandable support frame adapted for implantation within a body vessel from a delivery catheter. In one aspect, the support means is a support frame forming part of an implantable valve. In another aspect, the support means can include an outer sleeve support frame, such as a hoop member.

The implantable frame preferably defines a substantially cylindrical or elliptical lumen providing a conduit for fluid flow. The frame structure may comprise a plurality of struts, which can be of any suitable structure or orientation. In some embodiments, the frame comprises a plurality of struts connected by alternating bends. For example, the frame can be a ring or annular tube member comprising a series of struts in a “zig-zag” pattern. The frame can also comprise multiple ring members with struts in a “zig-zag” pattern, for example by connecting the ring members end to end, or in an overlapping fashion. In some embodiments, the struts are substantially aligned along the surface of a tubular plane, and substantially parallel to the longitudinal axis of the support frame. Support frames can also be formed from braided strands of one or more materials, helically wound strands, ring members, consecutively attached ring members, tube members, and frames cut from solid tubes. Alternatively, the medical device can be an implantable valve comprising a frame member shaped in a serpentine configuration having a plurality of bends defining two or more legs, with a leaflet attached to each leg.

The support frame can have any suitable configuration and size. The support frame can be sized so that the second, expanded configuration is slightly larger in diameter that the inner diameter of the vessel in which the medical device will be implanted. This sizing can facilitate anchoring of the medical device within the body vessel and maintenance of the medical device at a point of treatment following implantation. Preferably, the support frame is configured for implantation in a body vessel having an inner diameter of about 5 mm to about 25 mm, more preferably about 8 mm to about 20 mm.

The implantable frame may be formed from any suitable biocompatible material that allows for desired therapeutic effects upon implantation in a body vessel. Examples of suitable materials include, without limitation, any suitable metal or metal alloy, such as: stainless steels (e.g., 316, 316L or 304), nickel-titanium alloys including shape memory or superelastic types (e.g., nitinol or elastinite); inconel; noble metals including copper, silver, gold, platinum, palladium and iridium; refractory metals including Molybdenum, Tungsten, Tantalum, Titanium, Rhenium, or Niobium; stainless steels alloyed with noble and/or refractory metals; magnesium; amorphous metals; plastically deformable metals (e.g., tantalum); nickel-based alloys (e.g., including platinum, gold and/or tantalum alloys); iron-based alloys (e.g., including platinum, gold and/or tantalum alloys); cobalt-based alloys (e.g., including platinum, gold and/or tantalum alloys); cobalt-chrome alloys (e.g., elgiloy); cobalt-chromium-nickel alloys (e.g., phynox); alloys of cobalt, nickel, chromium and molybdenum (e.g., MP35N or MP20N); cobalt-chromium-vanadium alloys; cobalt-chromium-tungsten alloys; platinum-iridium alloys; platinum-tungsten alloys; magnesium alloys; titanium alloys (e.g., TiC, TiN); tantalum alloys (e.g., TaC, TaN); L605; bioabsorbable materials, including magnesium; or other biocompatible metals and/or alloys thereof. Preferably, the implantable frame comprises a self-expanding nickel titanium (NiTi) alloy material, stainless steel or a cobalt-chromium alloy. The nickel titanium alloy sold under the tradename Nitinol.

Preferably, the frame material is preferably a self-expanding material capable of significant recoverable strain to assume a low profile for delivery to a desired location within a body lumen. After release of the compressed self-expanding resilient material, it is preferred that the frame be capable of radially expanding back to its original diameter or close to its original diameter. Accordingly, some embodiments provide frames made from material with a low yield stress (to make the frame deformable at manageable balloon pressures), high elastic modulus (for minimal recoil), and is work hardened through expansion for high strength. Particularly preferred materials for self-expanding implantable frames are shape memory alloys that exhibit superelastic behavior, i.e., are capable of significant distortion without plastic deformation. Frames manufactured of such materials may be significantly compressed without permanent plastic deformation, i.e., they are compressed such that the maximum strain level in the resilient material is below the recoverable strain limit of the material. Discussions relating to nickel titanium alloys and other alloys that exhibit behaviors suitable for frames can be found in, e.g., U.S. Pat. No. 5,597,378 (Jervis) and WO 95/31945 (Burmeister et al.). A preferred shape memory alloy is Ni—Ti, although any of the other known shape memory alloys may be used as well. Such other alloys include: Au—Cd, Cu—Zn, In—Ti, Cu—Zn—Al, Ti—Nb, Au—Cu—Zn, Cu—Zn—Sn, CuZn—Si, Cu—Al—Ni, Ag—Cd, Cu—Sn, Cu—Zn—Ga, Ni—Al, Fe—Pt, U—Nb, Ti—Pd—Ni, Fe—Mn—Si, and the like. These alloys may also be doped with small amounts of other elements for various property modifications as may be desired and as is known in the art. Nickel titanium alloys suitable for use in manufacturing implantable frames can be obtained from, e.g., Memory Corp., Brookfield, Conn. One suitable material possessing desirable characteristics for self-expansion is Nitinol, a Nickel-Titanium alloy that can recover elastic deformations of up to 10 percent. This unusually large elastic range is commonly known as superelasticity.

The medical device can optionally comprise a bioabsorbable material. The biodegradable material, or combination of materials, can be chosen to provide desired characteristics upon implantation at a desired point of treatment, such as a desired time for absorption. For example, a biodegradable material can be chosen to degrade or be absorbed within a body over a period of weeks or months. Certain biodegradable polymers are known to degrade within the body at differing rates based upon the polymer selected and the point of implantation. Optionally, an implantable frame can comprise a core layer of a metal base material coated with a bioabsorbable material, such that absorption of the bioabsorbable material changes the flexibility of the frame after a desirable period of implantation in a body vessel. In some embodiments, a frame comprises a biostable core or “base” material surrounded by, or combined, layered, or alloyed with a bioabsorbable material.

Preferably, a bioabsorbable, biocompatible polymer is approved for use by the U.S. Food and Drug Administration (FDA). These FDA-approved materials include polyglycolic acid (PGA), polylactic acid (PLA), Polyglactin 910 (comprising a 9:1 ratio of glycolide per lactide unit, and known also as VICRYL™), polyglyconate (comprising a 9:1 ratio of glycolide per trimethylene carbonate unit, and known also as MAXON™), and polydioxanone (PDS). In general, these materials biodegrade in vivo in a matter of months, although some more crystalline forms can biodegrade more slowly. Optionally, one or more of the biodegradable polymers can be cross-linked by any suitable method to form a hydrogel biodegradable material. Other suitable biodegradable materials include: poly-alpha hydroxy acids (including polyactic acid or polylactide, polyglycolic acid, or polyglycolide), poly-beta hydroxy acids (such as polyhydroxybutyrate or polyhydroxyvalerate), epoxy polymers (including polyethylene oxide (PEO)), polyvinyl alcohols, polyesters, polyorthoesters, polyamidoesters, polyesteramides, polyphosphoesters, and polyphosphoester-urethanes. Naturally occurring polymers can also be used in or on the medical device, including: fibrin, fibrinogen, elastin, casein, collagens, chitosan, extracellular matrix (ECM), carrageenan, chondroitin, pectin, alginate, alginic acid, albumin, dextrin, and phosphorylcholine, as well as co-polymers and derivatives thereof. Various cross linked polymer hydrogels can also be used in forming the medical device, such as portions of the frame or coating on the frame.

Optionally, the surface of the support frame can be modified to promote desired processes, such as adhesion of a thromboresistant material or ingrowth of tissue inside a body cavity. For example, the surface of the frame can be roughened by grit blasting, chemical etching or electropolishing or any other technique known in the art to roughen the frame surface.

Bioactive Agents

A thromboresistant bioactive agent can be included in any suitable part of an implantable medical device. Selection of the type of thromboresistant bioactive, the portions of the medical device comprising the thromboresistant bioactive agent, and the manner of attaching the thromboresistant bioactive agent to the medical device can be chosen to perform a desired therapeutic function upon implantation. For example, a therapeutic bioactive agent can be combined with a biocompatible polyurethane, impregnated in an extracellular matrix material, incorporated in an implantable support frame or coated over any portion of the medical device. In one aspect, the implantable medical device can comprise one or more valve leaflets comprising a thromboresistant bioactive agent coated on the surface of the valve leaflet or impregnated in the valve leaflet. In another aspect, a thromboresistant bioactive material is combined with a biodegradable polymer or hydrogel (e.g., a polyethylene glycol and/or polyethylene oxide hydrogel) to form a portion of an implantable frame.

Medical devices comprising an antithrombogenic bioactive agent are particularly preferred for implantation in areas of the body that contact blood. An antithrombogenic bioactive agent is any therapeutic agent that inhibits or prevents thrombus formation within a body vessel. The medical device can comprise any suitable antithrombogenic bioactive agent. Types of antithrombotic bioactive agents include anticoagulants, antiplatelets, and fibrinolytics. Anticoagulants are bioactive agents which act on any of the factors, cofactors, activated factors, or activated cofactors in the biochemical cascade and inhibit the synthesis of fibrin. Antiplatelet bioactive agents inhibit the adhesion, activation, and aggregation of platelets, which are key components of thrombi and play an important role in thrombosis. Fibrinolytic bioactive agents enhance the fibrinolytic cascade or otherwise aid is dissolution of a thrombus. Examples of antithrombotics include but are not limited to anticoagulants such as thrombin, Factor Xa, Factor VIIa and tissue factor inhibitors; antiplatelets such as glycoprotein IIb/IIIa, thromboxane A2, ADP-induced glycoprotein IIb/IIIa, and phosphodiesterase inhibitors; and fibrinolytics such as plasminogen activators, thrombin activatable fibrinolysis inhibitor (TAFI) inhibitors, and other enzymes which cleave fibrin.

Further examples of antithrombotic bioactive agents include anticoagulants such as heparin, low molecular weight heparin, covalent heparin, synthetic heparin salts, coumadin, bivalirudin (hirulog), hirudin, argatroban, ximelagatran, dabigatran, dabigatran etexilate, D-phenalanyl-L-poly-L-arginyl, chloromethy ketone, dalteparin, enoxaparin, nadroparin, danaparoid, vapiprost, dextran, dipyridamole, omega-3 fatty acids, vitronectin receptor antagonists, DX-9065a, CI-1083, JTV-803, razaxaban, BAY 59-7939, and LY-51,7717; antiplatelets such as eftibatide, tirofiban, orbofiban, lotrafiban, abciximab, aspirin, ticlopidine, clopidogrel, cilostazol, dipyradimole, nitric oxide sources such as sodium nitroprussiate, nitroglycerin, S-nitroso and N-nitroso compounds; fibrinolytics such as alfimeprase, alteplase, anistreplase, reteplase, lanoteplase, monteplase, tenecteplase, urokinase, streptokinase, or phospholipid encapsulated microbubbles; and other bioactive agents such as endothelial progenitor cells or endothelial cells.

An antithrombotic agent, such as, heparin or a heparin derivative may be bound to the valve leaflet by any suitable method including physical, ionic, or covalent bonding, for example by applying solution of heparin or a heparin derivative to the valve leaflet surface or by dipping the valve leaflet in the solution. In one embodiment, heparin is bound to the valve leaflet using a suitable crosslinking agent such as a polyepoxide or carbodiimide cross linking agent such as 1-ethyl-3-(3-dimethylaminopropyl)-carbodiimide hydrochloride (EDC). In multi-layer constructs, heparin or other agents can be applied to the layers individually before incorporation of the layer into the construct, after the layers are incorporated into the construct (e.g. coating a luminal surface of an inner tubular layer), or both. Heparin can also be applied using a benzalkonium heparin (BA-Hep) isopropyl alcohol solution. This procedure treats the collagen with an ionically bound BA-Hep complex. Other coating, bonding, and attachment procedures, which are known in the art can also be used. Flowable (e.g. injectable) biomaterials may incorporate heparin or one or more other anti-thrombogenic agents in soluble form and/or bound to any suspended particulate biomaterial within the formulation, which can be impregnated into or coated on portions of the valve leaflet, particularly around attachment points to the frame.

An antithrombogenic bioactive agent can be incorporated in or applied to portions of the implantable medical device by any suitable method that permits adequate retention of the bioactive agent material and the effectiveness thereof for an intended purpose upon implantation in the body vessel. The configuration of the bioactive agent on or in the medical device will depend in part on the desired rate of elution for the bioactive. Bioactive agents can be coated directly on the medical device surface or can be adhered to a medical device surface by means of a coating. For example, an antithrombotic bioactive agent can be blended with a biocompatible polyurethane polymer and spray or dip coated on the device surface. The bioactive agent material can diffuse through the porous coating layer. Multiple porous coating layers and or pore size can be used to control the rate of diffusion of the bioactive agent material. Alternatively, a valve comprising a porous biocompatible polyurethane can be soaked in a solution comprising one or more bioactive agents, thereby absorbing the bioactive agent. The solution can be removed from the pores of the biocompatible polyurethane, leaving a bioactive agent impregnated in the polyurethane pores.

Bioactive agents may be bonded to a valve leaflet material, a support frame, an outer sleeve, or an adhesion promoting body vessel contact region, either directly via a covalent bond or via a linker molecule which covalently links the bioactive agent and the coating layer. Alternatively, the bioactive agent may be bound to the coating layer by ionic interactions including cationic polymer coatings with anionic functionality on bioactive agent, or alternatively anionic polymer coatings with cationic functionality on the bioactive agent. Hydrophobic interactions may also be used to bind the bioactive agent to a hydrophobic portion of the coating layer. The bioactive agent may be modified to include a hydrophobic moiety such as a carbon based moiety, silicon-carbon based moiety or other such hydrophobic moiety. Alternatively, the hydrogen bonding interactions may be used to bind the bioactive agent to the coating layer.

The bioactive agent material can be posited on the surface of the medical device and a porous coating layer can be posited over the bioactive agent material. Referring again to the device 200 of FIG. 4, the outer sleeve 280 preferably includes at least one layer comprising a porous polyurethane material in contact with a second layer comprising a bioactive agent. The second layer can comprise a remodelable material or a biocompatible polyurethane, as well as a growth factor and/or a bioactive agent. Preferably, the inner surface 281 of the outer sleeve 280 of the device 200 in FIG. 4 comprises a porous polyurethane material over a layer comprising a suitable bioactive agent. The bioactive agent can diffuse through the porous polyurethane and into the body vessel, for example to locally deliver an antithrombogenic bioactive agent near the valve 210. A porous layer is preferably configured to permit diffusion of the bioactive agent from the medical device upon implantation within the body at a desirable elution rate. Prior to implantation in the body, the diffusion layer can be substantially free of the bioactive agent. Alternatively, the diffusion layer can comprise a bioactive agent within pores in the diffusion layer. The outer sleeve 280 can also be configured to release a bioactive from the outer surface of the device, by including a porous outer layer. Optionally, the porous layer can comprise a mixture of a biodegradable polymer and a bioactive positioned within pores of a biostable polymer of a diffusion layer. In another embodiment, the porous layer can comprise a mixture of a biodegradable polymer and a biostable polymer, configured to permit absorption of the biodegradable polymer upon implantation of the medical device to form one or more channels in the biostable polymer to permit an underlying bioactive agent to diffuse through the pores formed in the biostable polymer.

Methods of Manufacture

In a fifth embodiment, methods for making a prosthetic valve for placement within a body passage are also provided. Preferably, the prosthetic valve comprises a thromboresistant material. According to one preferred method, a solution comprising a dissolved thromboresistant material is sprayed and dried on a mandrel.

Preferably, the prosthetic valve comprises one or more portions formed from a biocompatible polyurethane material, including one or more valve leaflets. More preferably, an implantable valve comprises a portion of a valve orifice formed from a non-porous biocompatible polyurethane based polymer as described above and sold under the tradename THORALON.

The biocompatible polyurethane material can be attached to an implantable support frame by drying a solution of the dissolved thromboresistant material on a surface with a desired shape. The thromboresistant material can be formed by at least one of three methods: (1) spraying, (2) dipping or (3) casting of the biocompatible polyurethane solution, and drying the polymer around portions of a support frame. Alternatively, a dried sheet of biocompatible polyurethane material can be adhered to a support frame using an adhesive, sutures, UV-activated polymers, melting, or any suitable means of attachment providing a desirably durable attachment between the thromboresistant material and the implantable frame. Preferably, a solution of the dissolved thromboresistant material can be coated onto a portion of the frame and attached to the frame as the solution is dried.

Polyurethane Solution Preparation

A valve leaflet can be formed by spray coating a solution comprising a dissolved thromboresistant material in a volatile organic solvent is coated by spraying, dipping or casting and dried by removal of the organic solvent to form a portion of an implantable valve. The solution is preferably a polyurethane dissolved in a suitable solvent. A solution for forming non-porous THORALON can be made by mixing the polyetherurethane urea (BPS-215) and the surface modifying additive (SMA-300) in a solvent, such as dimethyl formamide (DMF), tetrahydrofuran (THF), dimethyacetamide (DMAC), or dimethyl sulfoxide (DMSO). The solution typically contains between about 1% and 5% by weight SMA-3000 for either spray or dip coating solutions. The composition can contain up to about 40 wt % BPS-215 polymer, and different levels of polymer within the range can be used to fine tune the viscosity needed for a given process. Preferably, a solution for spray coating contains less than about 25% by weight of BPS-215 and SMA-3000 in a DMAC solvent, with a viscosity of less than about 2,000 Cp. Typical spray solutions can include a 50:50 weight percentage blend of the DMAC solvent and the pre-mixed solid composition comprising the BPS-215 and SAM-300. Solutions for dip coating can contain more BPS-215, having a viscosity of between about 2,000 and 3,000 Cp. The composition can contain less than 5 wt % polymer for some spray application embodiments. The entire composition can be cast as a sheet, or coated onto an article such as a mandrel or a mold.

A solution for forming porous THORALON can be made by mixing the polyetherurethane urea (BPS-215), the surface modifying additive (SMA-300) and micronized water soluble salt in a solvent a suitable solvent described above, preferably DMAC. The salt is preferably sodium chloride sieved at up to about 20-70 μm particle size. The amount of salt can be increased to increase the porosity of the polyurethane produced. Preferably, the weight of salt added to the solvent is about 5-15 times the amount of solid BPS-215 and SMA-300 added to the solvent, more preferably about 6-12 times. The solution typically contains between up to about 1% to 5% by weight SMA-3000 for either spray or dip coating solutions. The composition can contain up to about 40 wt % BPS-215 polymer, and different levels of polymer within the range can be used to fine tune the viscosity needed for a given process. Preferably, a solution for spray coating contains less than about 25% by weight of BPS-215 and SMA-3000 in a DMAC solvent, with a viscosity of less than about 2,000 Cp. Typical spray solutions can include a 50:50 weight percentage blend of the DMAC solvent and the pre-mixed solid composition comprising the BPS-215 and SAM-300. Solutions for dip coating can contain more BPS-215, having a viscosity of between about 2,000 and 3,000 Cp. The composition can contain less than 5 wt % polymer for some spray application embodiments. The entire composition can be cast as a sheet, or coated onto an article such as a mandrel or a mold.

Mandrel-Frame Spray Coating

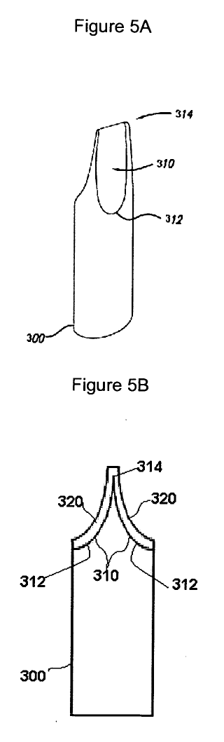

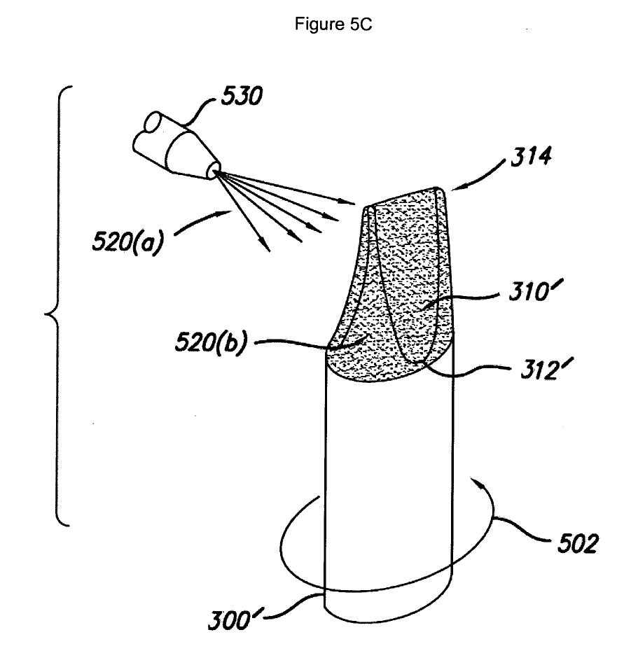

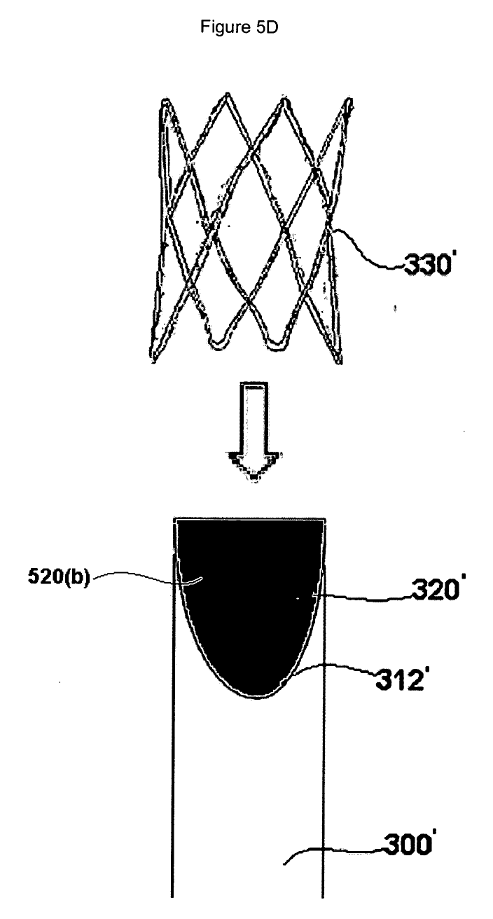

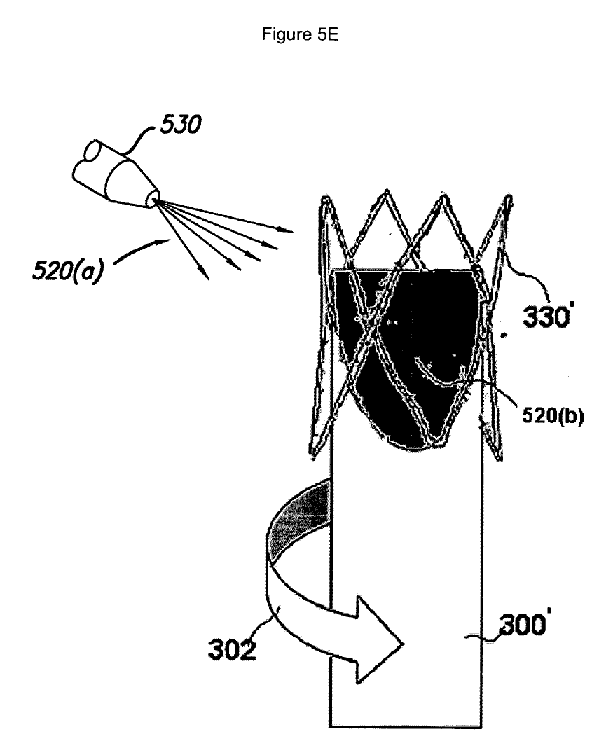

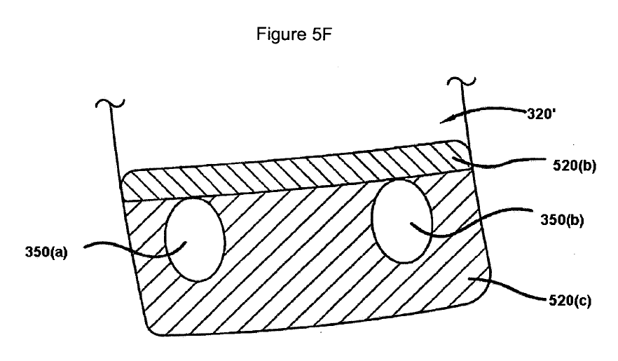

In a first aspect, a valve is formed by spray coating the solution of polyurethane in a suitable solvent onto a mandrel. Prior to spray coating or dip coating, a suitable mandrel surface is provided. The mandrel is preferably configured to provide a desirable leaflet shape. FIG. 5A shows a mandrel 300 for forming a valve leaflet. The mandrel 300 comprises a deposition surface 310 with a curved and tapered dimension leading to the distal end 314 of the mandrel 300. The edge 312 of the deposition surface 310 is shaped to conform to the desired configuration of a valve leaflet. The mandrel 300 can be made from any suitable material that permits the thromboresistant material to coated, dried on and removed from the mandrel surface. Suitable materials include stainless steel and glass. Preferably, at least a portion of the outer surface of the mandrel is formed in the desired shape of a valve leaflet. The leaflet can be formed by coating a thin layer of a solution of the thromboresistant material onto the shaped portion of the mandrel, drying the coating of the thromboresistant coating on the mandrel surface, and carefully removing the dried layer of thromboresistant coating.

Optionally, the surface of the mandrel or frame can be roughened or comprise raised or ingrained patterns to form correspondingly unevenly shaped coatings. For instance, a dimpled sheet of thromboresistant material can be formed by spray or dip coating a solution of the thromboresistant material onto the dimpled or pitted mandrel surface and removing the resulting dried coating from the coating surface. Textured, patterned or perforated thromboresistant materials can be used, for example, to promote tissue ingrowth through the material within a body vessel or alter fluid flow dynamics in the blood vessel. For example, a venous valve can comprise a leaflet with a textured, rough or perforated surface that provides desirable flow dynamics or a small amount of retrograde flow.

Optionally, the surface of the support frame 330 can be roughened prior to spraying or dipping the frame with the polyurethane solution. The support frame 330 can be roughened or textured in any convenient manner, such as by etching. Preferably, however, the surface is roughened or textured by abrading, for example, by abrading with an abrasive grit comprising at least one of sodium bicarbonate (USP), calcium carbonate, aluminum oxide, colmanite (calcium borate), or other abrasive particulates. Such roughening or texturing is most easily carried out by placing the medical device on a mandrel 300 in a position such that abrasive grit delivered from a nozzle impinges on the surface. The initial surface of the base material prior to roughening or texturing may be smoother than the desired surface roughness, or it may be even rougher. The grit size and feed rate of the abrasive grit, the structure of the nozzle, the pressure at which the abrasive grit is delivered from the nozzle, the distance of the surface from the nozzle and the rate of relative movement of the medical device and the nozzle are all factors considered in optimizing the roughening process. For example, when the support frame 330 is stainless steel, the abrading step can be carried out with an abrasive grit having a particle size of about 5 microns to about 500 microns. More preferably, the abrading step is carried out with sodium bicarbonate (USP) having a nominal particle size of about 50 microns. Such abrading is preferably carried out with the abrasive grit delivered at a pressure under flow of about 5 to about 200 PSI (about 34 to about 1380 KPa), most preferably about 100 PSI (about 690 KPa). Such abrading is also preferably carried out with the sodium bicarbonate or other abrasive grit 24 delivered at a grit feed rate of about 1 to about 1000 g/min, most preferably about 10 to about 15 g/min. The carrier gas or propellant for delivery of the abrasive grit is preferably nitrogen, air or argon, and most preferably nitrogen, although other gases may be suitable as well. The distance from the outlet of the nozzle to the center of the mandrel 300 can be about 1 to about 100 mm. A preferred nozzle is the Comco Microblaster; when employed, the preferred distance from the outlet of the nozzle to the center of the mandrel 300 is about 5 to about 10 mm.

To form a valve leaflet, the mandrel surface is first contacted with the polyurethane solution to join the biocompatible polyurethane material to a suitable frame. Prosthetic valves can be formed by applying one or more layers of the solution of the dissolved thromboresistant material composition to a mandrel and/or to an assembly comprising an implantable support frame fitted over a mandrel, and then drying the applied solution to remove excess volatile solvent and to solidify the solution coating to form one or more portions of the prosthetic valve. When applied to a mandrel alone, the dried thromboresistant coating can be separated from the mandrel and attached to an implantable frame. Alternatively, an implantable frame can be fitted over a mandrel that has been pre-coated with a layer of the thromboresistant material, and additional layers of the dissolved thromboresistant material can be applied to the frame and pre-coated mandrel together. The additional layers can adhere to or combine with the pre-coating layer on the mandrel to surround portions of the implantable frame, thereby securing a portion of the coating of thromboresistant material to the enclosed portions of the implantable frame. FIG. 5B shows the mandrel of FIG. 5A rotated 90-degrees after deposition of a layer of a biocompatible polyurethane on the distal portion of the mandrel. The mandrel 300 includes a deposition surface 310 and is bounded by an edge 312 of the deposition surface 310. Optionally, the edge 312 can form a raised ridge portion to provide a valve leaflet with a curved leaflet edge. A coating 320 of a biocompatible polyurethane is deposited on at least the deposition surface 310, although other portions of the mandrel 300 surface can also be coated. Two deposition surfaces meet at the distal end 314 of the mandrel 300. The coating 320 can be applied by any suitable method, including spray coating or dip coating, as described below.

FIG. 5C schematically illustrates a preferred process for coating a solution of thromboresistant material 520a onto the distal end 314 of the mandrel 300′. As described above, the solution of thromboresistant material preferably comprises a suitable solvent, a biocompatible polyurethane and a surface modifying agent. The distal end 314 of the mandrel 300′ is preferably configured to provide a desirable leaflet shape along the edge 312′. The solution of thromboresistant material 520a can be a DMAC solution of non-porous THORALON sprayed from a spray gun 530 onto the mandrel 300′ to form a substantially uniform coating layer 520b over the tapered portion 310′. Preferably, the mandrel 300′ is rotated 502 during spraying process to promote uniform coating of the mandrel 300′. Any suitable rate of rotation can be used, but a rate of 1 rpm is preferred. The solution of thromboresistant material is coated onto the deposition surface 310′ of the distal end 314′ of the mandrel 300′ and dried to form an article of manufacture substantially conforming to the shape of the tapered portion 510. Optionally, one or more bioactive agents can be coated onto the mandrel with the thromboresistant material. The process of FIG. 5C can be used, for example, to form a frameless implantable valve 12, an implantable valve 100 or an implantable valve 80, as shown in FIGS. 1A -3B. To form the frameless implantable valve 12, a mandrel having a pair of tapered deposition surfaces 310′ on opposite sides of the mandrel 300′, conforming to the shape of the monocusp valve 12, can be used. After spray coating a layer of non-porous THORALON of a desired thickness, the THORALON coating is dried on the mandrel to evaporate the DMAC solvent, such as by radiative heating over a desired period of time. The dried THORALON sleeve can be separated from the mandrel, for example by gently injecting a small amount of water between the mandrel and the THORALON sleeve with a small needle.

Methods of manufacturing implantable valves comprising one or more leaflets attached to a support frame are also provided. One or more valve leaflets can be attached to a support frame by any suitable technique. Preferably, the valve leaflets comprise a biocompatible polyurethane thromboresistant material such as non-porous THORALON that is attached to the support frame by being formed around and encapsulating portions of the support frame. The valves 80 and 100 can be formed by placing a frame over the distal portion 314′ of the mandrel 300′ after drying one or more layers of the coated polyurethane material 520(b) on the deposition surfaces 310′. FIG. 5D shows the placement of a frame 330′ over the coated mandrel 300′ shown in FIG. 5C. Any suitable frame 330′ can be placed around one end of the mandrel 300′ to form a mandrel-frame assembly. After the deposition of the coating 520(b), a support frame 330′ can be placed over the coated mandrel 300′, such that the portion of the frame to be attached to the leaflet is positioned over at least a portion of the deposition surface 320′. Preferably, one end of the support frame 330′ is positioned near the edge 312′ of the coating 520(b) over the deposition surface 320′. FIG. 5E shows the spray coating of a second layer of the thromboresistant material solution over the radially expanded support frame 330′ after placement of the support frame 330′ over the coating 520(b) on a portion of the mandrel 300′. A second coating of a polyurethane material can readily attach to the coating 520(b) already present on the deposition surface 320′ of the mandrel 300′, thereby attaching the polyurethane material to the frame. Typically, the polyurethane material in the coating 520(c) adheres more readily to the mandrel coating 520(b) than to the frame. Accordingly, the spray coating of the frame 330′ placed over the coating 520(b) on pre-coated deposition surface 320′ causes the two layers 520(b) and 520(c) of polyurethane to fuse together, forming a valve leaflet attached to the frame by forming a “sandwich” enclosing the frame between two fused layers of polyurethane, as shown in the cross sectional view of FIG. 5F.

FIG. 5F shows a cross-sectional view of the deposition surface 320′ of the mandril 300′ coated with a pre-coating layer 520b of a non-porous THORALON thromboresistant material. A second layer 520(c) of the THORALON thromboresistant material has been coated around the frame strut portions 350(a) and 350(b), forming two separate fused layers can be joined with the portions of the implantable frame 350(a), 350(b). Portions of two longitudinal struts of implantable frame 550a, 550b are positioned on the outer surface of the pre-coating layer 520b. An outer layer of thromboresistant material 520(c) is coated onto and dried over the pre-coating layer 520b and around the portions of the implantable frame 550a, 550b. Optionally, one or more bioactive agents can be coated onto the mandril with the thromboresistant material. Upon heating, the two THORALON layers will fuse to form a single layer attached to the implantable frame. Preferably, the pre-coating layer 520b is first dried on the mandril, then the implantable frame is placed over the coated mandril, and finally second layer of non-porous THORALON thromboresistant material 520′ is spray coated over the implantable frame as a solution comprising a suitable solvent such as DMAC and the thromboresistant material. The solvent in the spray solution preferably partially solubilizes the pre-coating layer 520(b) so that one fused layer of thromboresistant material is formed from a fusion of the pre-coating layer 520(b) and the thromboresistant material 520′. The fused layer can encapsulate portions of the implantable frame and be solidified by evaporation of residual solvent, thereby joining the thromboresistant material to the frame. The residual solvent in the fused layer can be evaporated by heating the coated medical device on the mandrel.

The dissolved thromboresistant material can also be applied to the mandrel and/or frame by dipping a mandrel, an implantable frame, or an assembly comprising both the mandrel and the implantable frame in the solution of dissolved thromboresistant material. The mandrel 500 can be dipped into the solution of thromboresistant material 520(a) and then removed from the solution and dried to form the pre-coating of thromboresistant material 520(b). The assembly 501 comprising the implantable frame 550 and the pre-coated mandrel 500′ can also be dipped into the solution of thromboresistant material 520(a) to form the leaflets 520.

An adhesion promoting body vessel contact region can be formed by spraying solution comprising a porous polyurethane composition from a spray nozzle onto the mandrel-frame assembly. Preferably, the solution is directed only to localized regions of the implantable frame, such as edges positioned to contact the surface of a body vessel upon implantation. More preferably, the deposition of the solution is carefully controlled and localized to prevent deposition onto the surface of the leaflets. For example, the leaflets can be masked or otherwise shielded during the spraying of the solution. Alternatively, the outer edges where the leaflet is joined to the implantable frame can be coated with the solution, while shielding the interior and free edge portions of the leaflet from deposition of the solution. Optionally, a bioactive agent such as a thromboresistant bioactive agent or a tissue growth promoting bioactive agent is incorporated in the pores of the porous polyurethane material, for example by including the bioactive agent in the solution.

Electrostatic Spray Deposition

The spraying step can optionally be performed using an Electrostatic spray deposition (ESD), for example to form a frameless valve or a valve leaflet. During ESD, particles of the polyurethane in solution are electrostatically charged when leaving the nozzle of the spray gun, and the mandrel 300 is maintained in a grounded configuration to attract the charged particles from the sprayed solution of thromboresistant material. The solution of thromboresistant material is first dissolved in a solvent and then sprayed onto the mandrel using an ESD process. The ESD process generally depends on the principle that a charged particle is attracted towards a grounded target. Without being confined to any theory, the typical ESD process may be described as follows. The solution that is to be deposited on the mandrel is typically charged to several thousand volts (typically negative) and held at ground potential. The charge of the solution is generally great enough to cause the solution to jump across an air gap of several inches before landing on the target. As the solution is in transit towards the target, it fans out in a conical pattern which aids in a more uniform coating. In addition to the conical spray shape, the electrons are further attracted towards the metal portions of the target, rather than towards the non conductive base the target is mounted on, leaving the coating mainly on the target only.

Generally, the ESD method allows for control of the coating composition and surface morphology of the deposited coating. In particular, the morphology of the deposited coating may be controlled by appropriate selection of the ESD parameters, as set forth in WO 03/006180 (Electrostatic Spray Deposition (ESD) of biocompatible coatings on Metallic Substrates), incorporated herein by reference. For example, a coating having a uniform thickness and grain size, as well as a smooth surface, may be obtained by controlling deposition conditions such as deposition temperature, spraying rate, precursor solution, and bias voltage between the spray nozzle and the medical device being coated. The deposition of porous coatings is also possible with the ESD method.

One hypothetical example of an electrostatic spraying apparatus is provided. Specifically, a solution of a non-porous THORALON material could be loaded into a 20 mL syringe of an ESD apparatus from Teronics Development Corp., which can then be mounted onto a syringe pump and connected to a tub that carries the solution to a spray head. The syringe pump could then used to purge the air from the solution line and prime the line and spray nozzle with solution. An electrical connection to the nozzle supplied the required voltage. An implantable frame could then be slipped over a mandrel (Teronics Development Corp., 2 mm×30 mm) until one end of the implantable frame makes contact with the electrical connection at one end of the mandrel. This connection can be used to provide a grounding potential to the implantable frame. A motor could then activated to rotate the mandrel at a constant speed of about 1 rpm. The syringe pump could then be activated to supply the nozzle with a consistent flow of solution, and the power supply could be activated to provide a charge to the solution and cause the solution to jump the air gap and land on the surface of the implantable frame. As the coated surface is rotated away from the spray path, the volatile portion of the solution could be evaporated leaving a coating of therapeutic agent behind. The implantable frame could be continually rotated in the spray pattern until the desired amount of non-porous THORALON material accumulates. During the coating process, the implantable frame could preferably be kept at ambient temperature and humidity, the solution could be pumped at a rate of about 2-4 cm³/hr through the spray gun (which can be placed at a horizontal distance of approximately 6 cm from the stents), and the bias voltage between the spray nozzle and the mandrel-frame assembly should be approximately 10-17 kilovolts.

Dip Coating

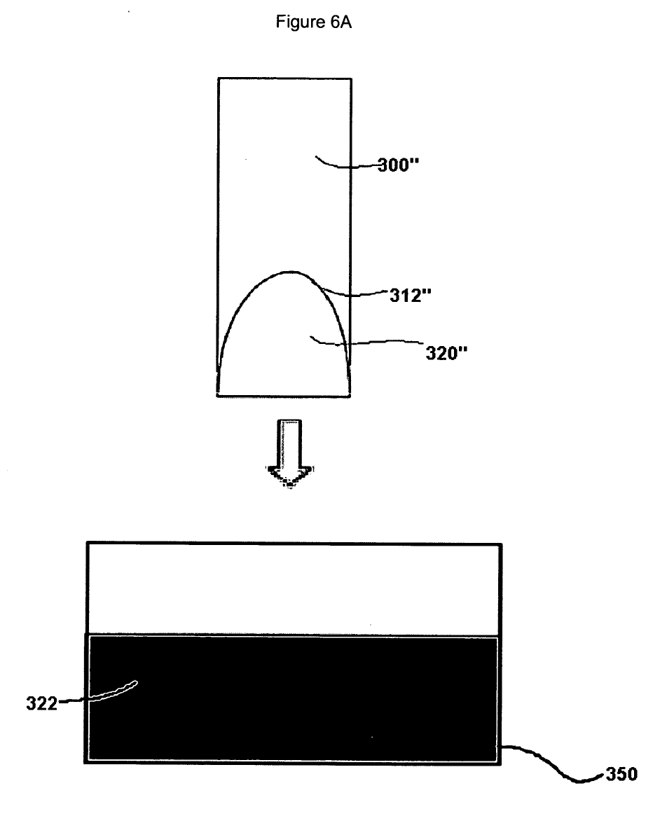

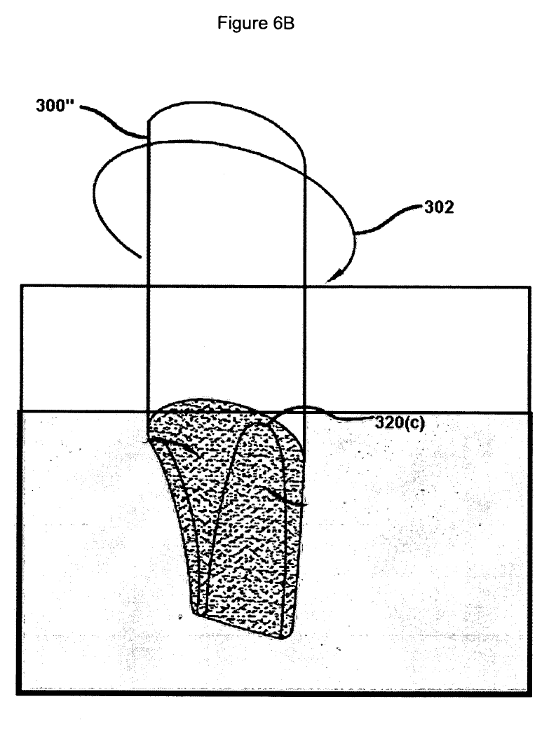

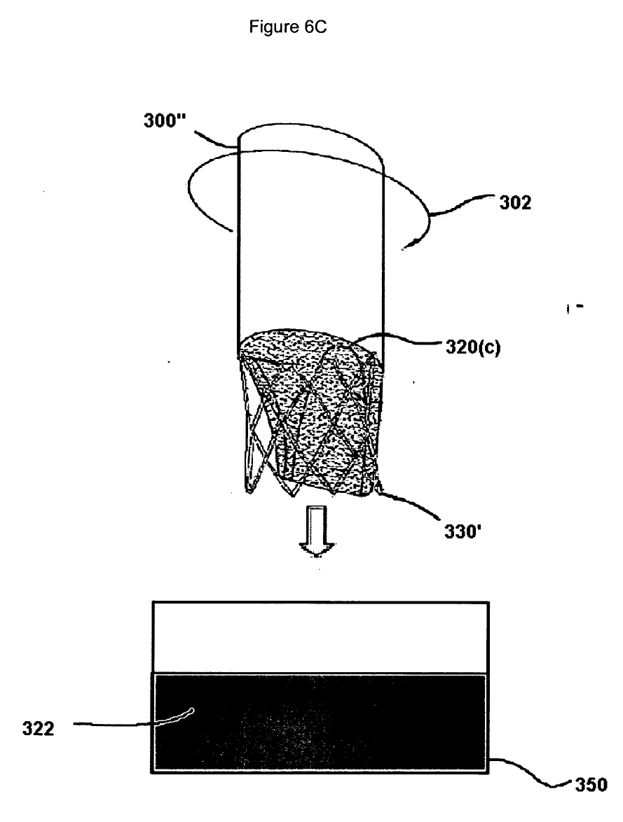

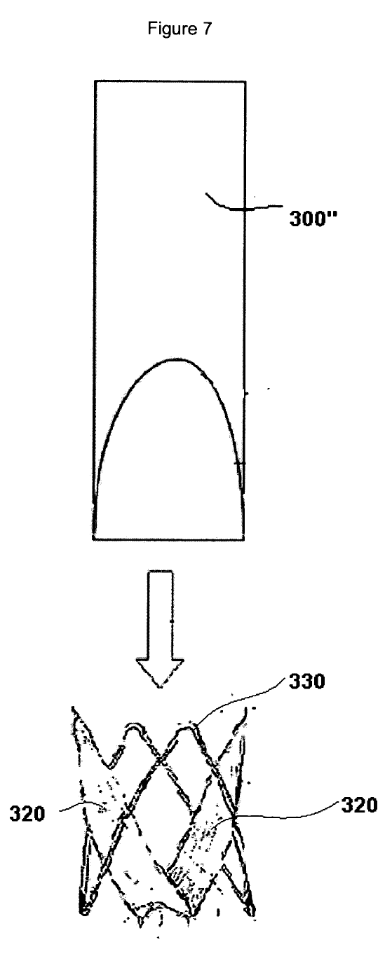

In another aspect of the second embodiment, a valve is formed by dip coating a solution of polyurethane in a suitable solvent onto a mandrel. As shown in FIG. 6A, a mandrel 300″ is dipped into a reservoir 350 containing a solution 322 comprising the thromboresistant material and a suitable solvent, as described above. Typically, the density and/or viscosity of the thromoresistant material in a solution 322 for dipping application than for spraying. The solvent may include dimethyl formamide (DMF), tetrahydrofuran (THF), dimethyacetamide (DMAC), or dimethyl sulfoxide (DMSO), or mixtures thereof. The composition can contain from about less than 1 wt % to about 40 wt % polyurethane polymer, and different levels of polymer within the range can be used to fine tune the viscosity needed for a given process. The solution 322 desirably comprises about 5 to about 25 wt % polymer. To form a porous valve leaflet, water-soluble salt particulates can be mixed into the solution 322.

The coating surface on the mandrel and/or frame can optionally be heated before, during or after coating with the thromboresistant material. Preferably, the solution and coating surface are at a similar or the same temperature. The mandrel and solution can be maintained at any temperature that maintains the solution in a liquid state with a desired level of viscosity. For THORALON polyureaurethane materials, a mandrel temperature of about 50° to about 60° C. is preferred for the dip coating process, preferably about 55° C.

The dissolved thromboresistant material in the solution 322 can be applied to the mandrel and/or frame by dipping a mandrel 300″ as shown in FIG. 6B. First, one or more layers of the thromboresistant material are coated onto the deposition surface 320″ of the mandrel 300″ and dried thereon. The coating surface can be spun at any suitable rate before, during or after contact with the solution of thromboresistant material. The mandrel can be spun clockwise, counter-clockwise or the rotation can be reversed once or more at any point during the coating or drying process. For THORALON polyurethaneurea thromboresistant materials, the coating surface of a mandrel or assembly can be rotated between about 1 rpm to about 120 rpm in a clockwise direction going into the solution, and a counterclockwise direction during removal from the solution and during drying. The rate of rotation can depend on the viscosity of the solution. Generally, the higher the viscosity of the solution, the faster the mandrel is spun while in contact with the solution, to promote more uniformity in coating thickness over the coating surface. A slower rotation rate can be employed in a solution with a lower viscosity. The viscosity of the solution of thromboresistant material can be varied, depending on the desired composition of the material. Generally, solution viscosities of between about 200 to 20,000 centipoise are suitable for coating a mandrel or assembly, preferably between about 600 and 1,000 centipoise.

During the dipping process, the mandrel can be translated into the solution of thromboresistant material at any rate that promotes desirable properties of the coating of thromboresistant materials. The rate of translation into or out of the solution can be the same or different. For THORALON polyurethaneurea thromboresistant materials, preferred translation rates for movement of the coating surface into or out of the solution correspond movement of 1 inch of length of coating surface with respect to the surface of the solution in a time between about 2 to about 20 seconds, depending on the viscosity and composition of the solution. Preferably, the rate of translation of the coating surface is slower going into the solution and faster exiting the solution.

Optionally, the coating surface on the mandrel or frame can remain in the solution for a suitable dwell time. The coating surface can be stationary or can be rotated during all or part of the dwell time. For THORALON polyurethaneurea thromboresistant materials, preferred dwell times are between 1 second and 1 minute, while rotating the coating surface in the solution. When a coating surface is dipped multiple times in the solution, the coated surface of the mandrel, frame or assembly, is preferably briefly dried for an intermittent drying time of about 1 minute to about 1 hour, to remove some removing excess volatile solvent. For THORALON polyurethaneurea thromboresistant materials dissolved in dimethyacetamide solvent, the coating surface is preferably maintained at a drying temperature of about 40° C. to about 60° C. during the intermittent drying period. Although the coating surface can be heated, other embodiments provide dipping methods without heating of the coating surface.

Next, as shown in FIG. 6C, an implantable frame 330, is placed over the coated layer 320(c) of the thromboresistant material adhered to the deposition surface 320″ of the mandrel 300″ to form an assembly comprising the mandrel 300″, the layer of thromboresistant material 320(c) and the frame 330′ placed around the layer 320(c). The assembly is then dipped into the solution 322 in the reservoir 350 to deposit a second layer of the thromboresistant material over the assembly. Typically, the thromboresistant material will adhere more readily to the layer 320(c) of the thromboresistant material than to the frame 330″. Accordingly, two layers of the thromboresistant material are fused around the frame, thereby joining the thromoresistant material to the frame. Preferably, the edges of attachment between the frame and the valve leaflet are reinforced by injecting a small amount of a solution 322 comprising the thromboresistant material in a suitable solvent into the space between the frame and the valve leaflet.

After applying the final coat of the solution, and removal of the coating and removal from the solution, the coated surface of the mandrel, frame or assembly, is preferably dried by removing excess volatile solvent. The coated surface can be dried in a heat chamber, and maintained at a suitable temperature for a suitable period of time to remove excess solvent and dry the coating. The drying temperature can be set suitably high to evaporate excess solvent from the coating, and can depend on the solvent used in the solution. Preferably, the drying temperature is substantially the same as the temperature of the solution and/or the mandrel. For THORALON polyurethaneurea thromboresistant materials dissolved in dimethyacetamide solvent, the final medical device comprising the THORALON material attached to a frame is preferably maintained at a drying temperature of about 40° C. to about 60° C. for a period of between about 1 minute to about 24 hours to evaporate, more preferably between about 1 hour and 24 hours. Finally, as shown in FIG. 7, the mandrel 300″ can be removed from the solution and dried to form a valve 332 comprising a pair of leaflets 320 attached to the frame 330″. Preferably, the valve 332 can be treated to remove any residual solvent, as needed. For example, the valve 332 can be placed in a water bath to remove trace amounts of DMAC solvent. Preferably, the level of DMAC in the valve leaflet is reduced to about 1090 ppm or lower, more preferably less than about 100 ppm. Residual levels of DMAC in a valve leaflet can be determined by any suitable method, including assays comprising the step of contacting the leaflet with sodium sulfate.

Alternative Leaflet Attachment Methods

Alternatively, one or more valve leaflets can be formed from a sheet of thromboresistant material attached to the frame by other methods. In one embodiment, a sheet of thromboresistant material is cut to form a leaflet and the edges of the leaflet are wrapped around portions of a support frame and portions of the thromboresistant material sealably connected together to fasten the thromboresistant material around the frame. For example, one edge of a sheet of thromboresistant material can be wrapped around a portion of the support frame and held against the body of the thromboresistant material, so that the thromboresistant material forms a lumen enclosing the support frame portion. A small amount of a suitable solvent is then applied to the edge of the thromboresistant material to dissolve the edge into an adjacent portion of the thromboresistant material and thereby seal the material around the support frame.

In another embodiment, the sheet of thromboresistant material is shaped to form a leaflet that is attached to a portion of a support frame using stitching through the thromboresistant material and around a portion of the support frame, adhesives, tissue welding or cross linking to directly join the thromboresistant material to the frame. A valve leaflet attached to a support frame can be permitted to move relative to the support frame, or the valve leaflet can be substantially fixed in its position or orientation with respect to the support frame by using attachment configurations that resist relative movement of the leaflet and the support frame.

Casting

A tubular sleeve of THORALON polyurethane material can be attached to a series of coaxially-aligned hoop members by casting. For example, the outer sleeve 280 of the medical device 200 shown in FIG. 4 can be manufactured by casting a layer of thromobresistant material along the interior surface of a tubular mold. The outer sleeve 280 is preferably formed by casting a biocompatible polyurethane material on the inner wall of a tubular mold. One or more sinusoidal hoop members 282 formed from a self-expanding biocompatible metal or metal alloy can be placed inside the tubular mold. Preferably, a first layer of polyurethane coating is first applied to the interior surface of the mold. Then the sinusoidal hoop members 282, or any suitable frame (such as a stent), is placed inside the coated mold. The hoop members 282 can be positioned at either end of the mold. Next, additional coating layers of the polyurethane material can be deposited over the sinusoidal members 282. The additional layers can join to the first layer to form a continuous outer sleeve 280 that surrounds the sinusoidal hoop members 282 in a “sandwich” manner. The sinusoidal hoop members 282 can be formed from a self-expanding material having an expanded state with a wider diameter than the interior diameter of the tubular mold, so as to exert a force in an outward radial direction after formation of the polyurethane coating to the device.

The polyurethane can be coated on the interior surface of the tubular mold by drying a polyurethane solution inside a suitable tubular mold (such as a quartz tube) while rotating the tubular mold to provide an outer sleeve structure. First, a suitable polyurethane solution, such as porous or non-porous THORALON, is prepared. The solution preferably has a weight ratio of solid (BPS-215 and SMA-300, and optionally containing a salt for forming the porous THORALON material) to DMAC of between about 1:1.5 to about 2:1. The solution can be coated on the inner surface of a glass tubular mold. The coated glass tube can be rotated at about 5 rpm along its longitudinal axis while being heated at a temperature and for a time sufficient to evaporate the solvent (e.g., about 40 deg. C. for about 2 hours). Optionally, hoop members or reinforcing elements (e.g., carbon fibers) can then be placed in contact with the dried coating inside the tube. Another layer of the solution can then be applied to the inside surface of the tube containing the hoop members or reinforcing elements. The glass tube is again heated and rotated to evaporate the solvent, leaving a casted structure having a tubular configuration and incorporating the hoop members or reinforcing elements within the polyurethane wall of the structure. The dried sleeve structure containing the hoop members can be removed from the glass tube and soaked in a warm water bath at a temperature of about 65 deg. C. for about 1 hour, then removed and dried. Optionally, a valve can be radially compressed, deployed and then secured within the lumen of the outer sleeve. The diameter of the tubular mold is preferably less than the maximum diameter of the fully expanded hoop member of valve. Preferably, the valve and hoop members are self-expanding structures that provide an outward radial force to provide shape and stability to the outer sleeve.

Medical Device Delivery and Methods of Treatment

The medical devices as described herein can be delivered to any suitable body vessel, including a vein, artery, biliary duct, ureteral vessel, body passage or portion of the alimentary canal. Methods for delivering a medical device as described herein to any suitable body vessel are also provided, such as a vein, artery, biliary duct, ureteral vessel, body passage or portion of the alimentary canal. While many preferred embodiments discussed herein discuss implantation of a medical device in a vein, other embodiments provide for implantation within other body vessels. In another matter of terminology there are many types of body canals, blood vessels, ducts, tubes and other body passages, and the term “vessel” is meant to include all such passages.

Preferably, the valve is implanted percutaneously to a point of treatment in a body vessel using any suitable delivery device, including delivery catheters dilators, sheaths, and/or other suitable endoluminal devices. Alternatively, the prosthetic valves can be placed in body vessels or other desired areas by any suitable technique, including percutaneous delivery as well as surgical placement. The valve advantageously has a radially compressed and a radially expanded configuration and can be implanted at a point of treatment within a body vessel by delivery and deployment with an intravascular catheter. The support frame can optionally provide additional function to the medical device. For example, the support frame can provide a stenting function, i.e., exert a radially outward force on the interior wall of a vessel in which the medical device is implanted. By including a support frame that exerts such a force, a medical device according to the invention can provide both a stenting and a valving function at a point of treatment within a body vessel.

One method of deploying the valve in a vessel involves radially compressing and loading the frame into a delivery device, such as a catheter. A restraining means may maintain the valve in the radially compressed configuration. For example, a self-expanding valve may be retained within a slideable sheath, while valves that are not self-expanding may be crimped over a balloon portion of a delivery catheter. The compressed valve is thereby mounted on the distal tip of the delivery device, translated through a body vessel on the delivery device, and deployed from the distal end of the delivery device. For example, a delivery device may be a catheter comprising a pushing member adapted to urge the valve away from the delivery catheter. A sheath may be longitudinally translated relative to the valve to permit the valve to radially self-expand at the point of treatment within a body vessel. Alternatively, a balloon may be inflated to radially expand the valve.

In some embodiments, medical devices having a frame with a compressed delivery configuration having a low profile with a small collapsed diameter and desired flexibility, may be able to navigate small or tortuous paths through a variety of body vessels. A low-profile medical device may also be useful in coronary arteries, carotid arteries, vascular aneurysms, and peripheral arteries and veins (e.g., renal, iliac, femoral, popliteal, sublavian, aorta, intercranial, etc.). Other nonvascular applications include gastrointestinal, duodenum, biliary ducts, esophagus, urethra, reproductive tracts, trachea, and respiratory (e.g., bronchial) ducts. These applications may optionally include a sheath covering the medical device. Other methods further comprise the step of implanting one or more frames attached to one or more valve members. Preferably, the medical devices described herein are implanted from a portion of a catheter inserted in a body vessel.

Still other embodiments provide methods of treating a subject, which can be animal or human, comprising the step of implanting one or more support frames as described herein. Methods of treatment preferably comprise the step of implanting one or more frames attached to one or more valve members, as described herein. In some embodiments, methods of treating may also include the step of delivering a medical device to a point of treatment in a body vessel, or deploying a medical device at the point of treatment. Methods for treating certain conditions are also provided, such as venous valve insufficiency, varicose veins, esophageal reflux, restenosis or atherosclerosis. In some embodiments, the invention relates to methods of treating venous valve-related conditions.

A “venous valve-related condition” is any condition presenting symptoms that can be diagnostically associated with improper function of one or more venous valves. In mammalian veins, venous valves are positioned along the length of the vessel in the form of leaflets disposed annularly along the inside wall of the vein which open to permit blood flow toward the heart and close to prevent back flow. These venous valves open to permit the flow of fluid in the desired direction, and close upon a change in pressure, such as a transition from systole to diastole. When blood flows through the vein, the pressure forces the valve leaflets apart as they flex in the direction of blood flow and move towards the inside wall of the vessel, creating an opening therebetween for blood flow. The leaflets, however, do not normally bend in the opposite direction and therefore return to a closed position to restrict or prevent blood flow in the opposite, i.e. retrograde, direction after the pressure is relieved. The leaflets, when functioning properly, extend radially inwardly toward one another such that the tips contact each other to block backflow of blood. Two examples of venous valve-related conditions are chronic venous insufficiency and varicose veins.

In the condition of venous valve insufficiency, the valve leaflets do not function properly. For example, the vein can be too large in relation to the leaflets so that the leaflets cannot come into adequate contact to prevent backflow (primary venous valve insufficiency), or as a result of clotting within the vein that thickens the leaflets (secondary venous valve insufficiency). Incompetent venous valves can result in symptoms such as swelling and varicose veins, causing great discomfort and pain to the patient. If left untreated, venous valve insufficiency can result in excessive retrograde venous blood flow through incompetent venous valves, which can cause venous stasis ulcers of the skin and subcutaneous tissue. Venous valve insufficiency can occur, for example, in the superficial venous system, such as the saphenous veins in the leg, or in the deep venous system, such as the femoral and popliteal veins extending along the back of the knee to the groin.

The varicose vein condition consists of dilatation and tortuosity of the superficial veins of the lower limb and resulting cosmetic impairment, pain and ulceration. Primary varicose veins are the result of primary incompetence of the venous valves of the superficial venous system. Secondary varicose veins occur as the result of deep venous hypertension which has damaged the valves of the perforating veins, as well as the deep venous valves. The initial defect in primary varicose veins often involves localized incompetence of a venous valve thus allowing reflux of blood from the deep venous system to the superficial venous system. This incompetence is traditionally thought to arise at the saphenofemoral junction but may also start at the perforators. Thus, gross saphenofemoral valvular dysfunction may be present in even mild varicose veins with competent distal veins. Even in the presence of incompetent perforation, occlusion of the saphenofemoral junction usually normalizes venous pressure.

The initial defect in secondary varicose veins is often incompetence of a venous valve secondary to hypertension in the deep venous system. Since this increased pressure is manifested in the deep and perforating veins, correction of one site of incompetence could clearly be insufficient as other sites of incompetence will be prone to develop. However, repair of the deep vein valves would correct the deep venous hypertension and could potentially correct the secondary valve failure. Apart from the initial defect, the pathophysiology is similar to that of varicose veins.

The invention includes other embodiments within the scope of the claims, and variations of all embodiments. Additional understanding of the invention can be obtained by referencing the detailed description of embodiments of the invention, below, and the appended drawings.

### INDUSTRIAL APPLICABILITY

Among other applications, the present invention can be used for providing a medical implantable device such as a valve for implantation within a human or veterinary patient, and therefore finds applicability in human and veterinary medicine.

It is to be understood, however, that the above-described device is merely an illustrative embodiment of the principles of this invention, and that other devices and methods for using them may be devised by those skilled in the art, without departing from the spirit and scope of the invention, It is also to be understood that the invention is directed to embodiments both comprising and consisting of the disclosed parts.

### EXAMPLES

### Example 1

### Compositions for Coating a Support Frame with a Non-THORALON Biocompatible Polyurethane

A solution for spray coating non-porous THORALON can be made by mixing 25 g of a solid mixture containing polyetherurethane urea (BPS-215) and 2% wt of the surface modifying additive (SMA-300) in dimethyacetamide (DMAC) solvent for a total solution weight of 100 g. The solution has a viscosity of less than about 2,000 Cp. The entire composition can be cast as a sheet, or coated onto an article such as a mandrel or a mold. To prepare a solution for spray coating porous THORALON, the micronized (ca. 25 mm) sodium chloride salt in an amount equal to about six times the total solid weight of the BPS-215 and SMA-300 components can be added to the solution.

A solution for dip coating non-porous THORALON can be made by mixing 25 g of a solid mixture containing polyetherurethane urea (BPS-215) and 5% wt of the surface modifying additive (SMA-300) in dimethyacetamide (DMAC) solvent for a total solution weight of 100 g. The solution has a viscosity of about 2,000 Cp. To prepare a solution for spray coating porous THORALON, the micronized (ca. 25 mm) sodium chloride salt in an amount equal to about six times the total solid weight of the BPS-215 and SMA-300 components can be added to the solution. The entire composition can be cast as a sheet, or coated onto an article such as a mandrel or a mold.

### Example 2

### Coating a Support Frame with a THORALON Biocompatible Polyurethane

An implantable prosthetic valve was prepared by spraying a self-expanding Nitinol alloy stent with a THORALON material solution. A non-porous THORALON spray solution described in Example 1 was sprayed at a rate of about 1 mL/min from a 0.028-inch spray gun nozzle at a distance of about 6 inches from the surface of the stent. The coated stent was heated at about 40 deg. C. for about 20 minutes between application of each spray coating to form the valve leaflets on the frame. A valve leaflet having a thickness of about 0.0025-inch was deposited after 7-8 spray coats on the mandrel. The THORALON material can be formulated to provide a porous or non-porous material by using the appropriate coating composition (see, e.g., Example 1). The following steps were followed to form the implantable prosthetic valve:
a. A Nitinol frame is cleaned with an organic solvent such as acetone or isopropyl alcohol to remove all traces of particulates and other surface contaminants.
b. A clean, glass mandrel that has the same diameter as the frame and is about 18 inches long is placed in a rotating holder. This holder fastens to both ends of the mandrel, positions the mandrel horizontally and rotates the mandrel around its longitudinal axis at about 1 revolution per second.
c. A solution of about 20% Thoralon in dimethylacetamide (DMAC) is prepared and shaken overnight at room temperature to achieve thorough mixing.
d. A portion of the Thoralon solution is loaded into an air sprayer.
e. The sprayer is activated and Thoralon solution applied to the rotating mandrel. Care is made to distribute the Thoralon uniformly in the horizontal direction so that the thickness of the Thoralon will be equal at every location on the mandrel.
f. After receiving the Thoralon spray, the coated mandrel is removed from the holder.
g. Rubber stoppers with center holes are attached to each end of the mandrel after which the mandrel is placed on a drying device that rotates the mandrel and radiates heat uniformly with respect to the mandrel. Filtered air also is passed over the mandrel during this time.
h. After about 2 hours the Thoralon coating is dry—having lost most of its DMAC to evaporation.
i. The rubber stoppers are then removed from the mandrel. The frame that is to be coated with Thoralon is carefully slid onto the mandrel and the mandrel is again placed in the holder.
j. Steps 5 through 8 are then repeated in order to apply a coating of Thoralon to the outside of the frame. Because of the DMAC present in this second Thoralon spray application, the layer of Thoralon located on the inside of the frame also becomes adhered to the frame.
k. The rubber stoppers are again removed from the mandrel.
l. A water/detergent solution is directed at one end of the mandrel at the boundary between the Thoralon and the glass tube.
m. The Thoralon will lift off from the glass tube and the frame and the Thoralon on the glass tube are subsequently slid off from the mandrel.
n. The frame and Thoralon are then rinsed with deionized water to remove the detergent.
o. The Thoralon is trimmed from the ends of the frame and the frame then placed in an oven at a temperature of 60 degrees Celsius overnight (16 hours). This is done in order to remove most of the DMAC and to allow the orientation of the polymer additive that provides the desired surface characteristics (cf. U.S. Pat. No. 4,675,361, column 11, lines 12-16, et al).
p. The frame is then removed from the oven and placed in deionized water for 4-6 hours at 60 degrees Celsius to remove any salt present (if a porous THORALON composition was used) and remaining traces of DMAC from the Thoralon.

### Example 3

### Coating a Support Frame with a Non-Porous THORALON Biocompatible Polyurethane

A venous valve comprising THORALON polyureaurethane thromboresistant valve leaflets was made by the following dipping process. First, a stainless steel mandrel can be heated to about 55° C. and spun clockwise at a rate of about 5 rpm. The spinning mandrel can be translated into a solution of thromboresistant material in DMAC solvent having a viscosity of between about 600 and 1,000 centipoise at a translation rate of about 1 inch per 5 seconds. The spinning mandrel can remain in the solution for a dwell time of about 10 seconds, before reversing the direction of rotation of the spinning mandrel and removing the mandrel from the solution at a rate of about 1 inch per 2 seconds. The coated mandrel can be dried for about 1 minute at about 60° C. and an implatable frame secured over the coating surface enclosing one end of the mandrel, thereby forming an assembly. The dipping procedure for the mandrel can be repeated for the assembly one or more times until the valve leaflets have a desired thickness, thereby forming a valve by attaching a pair of leaflets comprising the thromboresistant polyureaurethane material to the frame. After the coating and dipping processes are completed, the frame/valve can be dried for about 8 hours at a temperature of about 60° C. to remove excess solvent and to solidify the leaflets and the leaflet attachment to the frame. After drying, the valve can be removed from the mandrel, for example by inserting a fine gauge needle between the valve leaflet and the mandrel coating surface and injecting a small volume of water to promote separation of the valve leaflet from the mandrel.

### Example 4

### Cast Coating to Form a THORALON Biocompatible Polyurethane Sleeve

A tubular sleeve of THORALON polyurethane material attached to a series of coaxially-aligned hoop members was prepared. The following steps were followed to form the implantable prosthetic valve:
a. about 10 mL of a THORALON/DMAC polyurethane solution was prepared with a weight ratio of solid (BPS-215 and SMA-300, and optionally containing a salt for forming the porous THORALON material) to DMAC of between about 1:1.5 to about 2:1;
b. a glass tube was cleaned with soap and water, and about 2 mL of the solution was applied uniformly to the inside of the glass tube;
c. the coated glass tube was heated while rotating the tube slowly about the longitudinal axis (ca. 5 rpm) for about 2 hours at about 40 deg. C.;
d. the coated glass tube is cooled to room temperature and multiple self-expanding hoop members were deployed within the coated glass tube;
e. about 2 mL of the solution was applied uniformly to the inside of the glass tube and around the hoop members;
f. the coated glass tube and hoop members was heated while rotating the tube slowly about the longitudinal axis (ca. 5 rpm) for about 2 hours at about 40 deg. C.;
g. the dried sleeve structure containing the hoop members was removed from the glass tube and soaked in a warm water bath at a temperature of about 65 deg. C. for about 1 hour, then removed and dried; and
h. a valve from example 2 was radially compressed and deployed within the lumen of the outer sleeve.

We claim:

1. An implantable valve comprising: a valve leaflet comprising a biocompatible polyurethane and a growth factor.

2. The valve of claim 1, where the growth factor is selected from the group consisting of: FGF1, FGF2, FGF3, FGF4, FGF5, FGF6, FGF7, FGF8, FGF9, FGF10, VEGF A, VEGF B, VEGF C, VEGF D, VEGF E, PIGF, PDGF, EGF, IFN-ALPHA, IFN-BETA, IFN-GAMMA, TGF-ALPHA, and TGF-BETA.

3. The valve of claim 1, where the valve leaflet further comprises a remodelable material.

4. The valve of claim 3, where the remodelable material is selected from the group consisting of: small intestine submucosa (SIS), renal capsule matrix (RCM) and urinary bladder matrix (UBM).

5. The valve of claim 3, where the valve leaflet comprises a first layer comprising the remodelable material and a second layer comprising the biocompatible polyurethane in contact with the remodelable material.

6. The valve of claim 3, where the valve leaflet comprises a first layer comprising the biocompatible polyurethane comprising an adhesion promoting body vessel contact region including a remodelable material in contact with a portion of the biocompatible polyurethane.

7. The valve of claim 1, where the valve leaflet comprises a free edge moveable between an open position and a closed position in response to fluid flow contacting the valve surface.

8. The valve of claim 1, where the valve leaflet further comprises a thromboresistant bioactive agent.

9. The valve of claim 8, where the thromboresistant bioactive agent is selected from the group consisting of: thrombin, Factor Xa, Factor VIIa, glycoprotein IIb/IIIa, thromboxane A2, ADP-induced glycoprotein Ib/IIIa, plasminogen activators, thrombin activatable fibrinolysis inhibitor (TAFI) inhibitors, heparin, low molecular weight heparin, covalent heparin, synthetic heparin salts, coumadin, bivalirudin (hirulog), hirudin, argatroban, ximelagatran, dabigatran, dabigatran etexilate, D-phenalanyl-L-poly-L-arginyl, chloromethy ketone, dalteparin, enoxaparin, nadroparin, danaparoid, vapiprost, dextran, dipyridamole, omega-3 fatty acids, vitronectin receptor antagonists, DX-9065a, CI-1083, JTV-803, razaxaban, BAY 59-7939, and LY-51,7717, eftibatide, tirofiban, orbofiban, lotrafiban, abciximab, aspirin, ticlopidine, clopidogrel, cilostazol, dipyradimole, sodium nitroprussiate, nitroglycerin, S-nitroso, alfimeprase, alteplase, anistreplase, reteplase, lanoteplase, monteplase, tenecteplase, urokinase, streptokinase, endothelial progenitor cells, phosphorylcholine, phosphatidylcholine, and endothelial cells.

10. The valve of claim 1, where the biocompatible polyurethane comprises a first layer comprising a base polymer and about 0.5% to about 5% of a surface modifying additive;
a. where the surface modifying additive comprises polydimethylsiloxane and the reaction product of diphenylmethane diisocyanate and 1,4-butanediol; and
b. where the base polymer is a polyetherurethane urea comprising polytetramethylene oxide and the reaction product of 4,4′-diphenylmethane diisocyanate and ethylene diamine.

11. The implantable valve of claim 1, where the valve leaflet has a thickness of between about 0.0001 inch and about 0.0050 inch.

12. The valve of claim 1, where the implantable valve further comprises a support frame and where the valve leaflet is attached to at least a portion of the support frame.

13. An implantable valve comprising a radially expandable support frame and two or more flexible valve leaflets comprising a biocompatible polyurethane, where each leaflet comprises a flexible leaflet free edge and at least two edges defining an adhesion promoting body vessel contact region and attached to the support frame, at least two of the leaflet free edges forming a valve orifice defined by at least two opposable flexible leaflet free edges, the valve orifice permitting fluid to flow in a first direction through the implantable valve when each valve leaflet is in the open position, each leaflet free edge being moveable in response to fluid flow contacting the leaflet free edge, and each adhesion promoting body vessel contact region comprising a second layer comprising a material selected from the group consisting of: a remodelable material, a growth factor, a bioactive agent and a porous biocompatible polyurethane.

14. The valve of claim 13, where the biocompatible polyurethane comprises a first layer comprising a base polymer and about 0.5% to about 5% of a surface modifying additive;
a. where the surface modifying additive comprises polydimethylsiloxane and the reaction product of diphenylmethane diisocyanate and 1,4-butanediol; and
b. where the base polymer is a polyetherurethane urea comprising polytetramethylene oxide and the reaction product of 4,4′-diphenylmethane diisocyanate and ethylene diamine.

15. The valve of claim 13, where the porous biocompatible polyurethane comprises a first layer comprising a base polymer and about 1% to about 5% of a surface modifying additive and a pore size of between about 10 μm and about 500 μm;
a. where the surface modifying additive comprises polydimethylsiloxane and the reaction product of diphenylmethane diisocyanate and 1,4-butanediol; and
b. where the base polymer is a polyetherurethane urea comprising polytetramethylene oxide and the reaction product of 4,4′-diphenylmethane diisocyanate and ethylene diamine.

16. The implantable valve of claim 13, where the adhesion promoting body vessel contact region comprises a porous biocompatible polyurethane having a void-to-volume ratio, preferably from about 0.40 to about 0.90.

17. The implantable valve of claim 13, where the implantable valve comprises two leaflets.

18. The valve of claim 13, where the thromboresistant bioactive agent is selected from the group consisting of: thrombin, Factor Xa, Factor VIIa, glycoprotein IIb/IIIa, thromboxane A2, ADP-induced glycoprotein IIb/IIIa, plasminogen activators, thrombin activatable fibrinolysis inhibitor (TAFI) inhibitors, heparin, low molecular weight heparin, covalent heparin, synthetic heparin salts, coumadin, bivalirudin (hirulog), hirudin, argatroban, ximelagatran, dabigatran, dabigatran etexilate, D-phenalanyl-L-poly-L-arginyl, chloromethy ketone, dalteparin, enoxaparin, nadroparin, danaparoid, vapiprost, dextran, dipyridamole, omega-3 fatty acids, vitronectin receptor antagonists, DX-9065a, CI-1083, JTV-803, razaxaban, BAY 59-7939, and LY-51,7717, eftibatide, tirofiban, orbofiban, lotrafiban, abciximab, aspirin, ticlopidine, clopidogrel, cilostazol, dipyradimole, sodium nitroprussiate, nitroglycerin, S-nitroso, alfimeprase, alteplase, anistreplase, reteplase, lanoteplase, monteplase, tenecteplase, urokinase, streptokinase, endothelial progenitor cells, phosphorylcholine, phosphatidylcholine, and endothelial cells.

19. The valve of claim 1, where the growth factor is selected from the group consisting of: FGF1, FGF2, FGF3, FGF4, FGF5, FGF6, FGF7, FGF8, FGF9, FGF10, VEGF A, VEGF B, VEGF C, VEGF D, VEGF E, PIGF, PDGF, EGF, IFN-ALPHA, IFN-BETA, IFN-GAMMA, TGF-ALPHA, and TGF-BETA.

20. An implantable valve comprising: a valve leaflet comprising a biocompatible polyurethane and an extracellular matrix material comprising at least one growth factor.
